(19) United States
(12) Patent Application Publication
(10) Pub. No.: US 2015/0010947 A1
(43) Pub. Date: Jan. 8, 2015

(54) Domain Swapping Modules

(71) Applicant: The Research Foundation For The State University Of New York, Syracuse, NY (US)

(72) Inventors: Stewart Loh, Manlius, NY (US); Jeung-hio Ha, Manlius, NY (US); Diana Mitrea, Memphis, TN (US)

(73) Assignee: The Research Foundation For The State University Of New York, Syracuse, NY (US)

(21) Appl. No.: 14/369,408

(22) PCT Filed: Dec. 27, 2012
(86) PCT No.: PCT/US2012/071789

§ 371 Date: Jun. 27, 2014

Related U.S. Application Data
(60) Provisional application No. 61/580,421, filed on Dec. 27, 2011.

(51) Int. Cl. C12N 9/96; C07K 14/435

(52) U.S. Cl. CPC C12N 9/96; C07K 14/435; C12Y 302/01021; C12Y 302/01004; C07K 2319/70
USPC 435/69.7; 530/350; 536/23.4; 530/402; 435/188; 536/23.2

(57) ABSTRACT

Methods and systems for creating a genetic construct, a protein, and a polymer comprising a domain swapping module. A domain swapping module is a fusion protein in which a lever protein, which has a long amino (N) to carboxy (C) terminal distance, is inserted into a surface loop of an assembler protein, thereby stretching the assembler protein and splitting it into two fragments held apart by the lever so that they cannot rejoin. If the assembler protein is split at the proper location, the fragments will recombine with their respective counterparts from either one or more different—but similarly-split—assembler proteins.

### CROSS-REFERENCE TO RELATED APPLICATIONS

This application claims priority to U.S. Provisional Patent Application Ser. No. 61/580,421, filed on Dec. 27, 2011 and entitled “Domain Swapping Modules,” the entire disclosure of which is incorporated herein by reference.

### BACKGROUND

This specification relates to the field of molecular biology and, more specifically, to methods and systems for utilizing or constructing domain swapping modules.

Domain swapping is a mechanism for forming protein dimers and oligomers with high specificity. It is distinct from other forms of oligomerization in that the binding interface is formed by reciprocal exchange of polypeptide segments. Swapping plays a physiological role in protein-protein recognition and it can also potentially be exploited as a mechanism for controlled self assembly. Previous experiments have demonstrated that domain-swapped interfaces can be engineered by inserting one protein into a surface loop of another protein. The key to facilitating a domain swap is to destabilize the protein when it is monomeric but not when it is oligomeric. This condition is achieved by employing the ‘mutually exclusive folding’ design to apply conformational stress to the monomeric state. Engineered, swapped proteins have the potential to be used to fabricate ‘smart’ biomaterials, or as binding modules from which to assemble heterologous, multi-subunit protein complexes.

Nature uses 3D domain swapping to mediate protein-protein recognition when high specificity is required. For example, cell surface-expressed cadherins are responsible for cell-cell adhesion and the development of tissue architecture. By binding via β-strand exchange between monomers, classical cadherins ensure that cells expressing a particular cadherin will adhere to cells expressing the same cadherin subtype and not another. The strictest definition of a domain swap requires that both monomeric and oligomeric states of the protein be observed, a polypeptide segment of the monomer exchange with the same segment of another monomer, and the structures of the monomer and oligomer be identical except at the points of strand exchange. This interaction can result in dimers, closed ring-shaped oligomers, or polymers of indefinite length. Indeed, runaway swapping occurs in protein deposition diseases such as prion amyloidosis and serpinopathies. Aside from its pathogenic manifestations, however, swapping can potentially be exploited as a mechanism for controlled self assembly. It has been suggested that homopolymeric hydrogels with targeted bulk properties can be fabricated from domain-swapped proteins or peptides. Taking this idea one step further, it may be possible to use domain swapping as a means to create self-assembling macromolecular structures of defined subunit composition, stoichiometry, and quaternary structure; e.g. multi-subunit enzyme complexes that efficiently catalyze otherwise difficult reactions. For this application domain-swapping proteins could serve as genetically-encoded tags, fused to the target subunits, to facilitate assembly of the heterologous complex.

### BRIEF SUMMARY

Materials created from domain swapping module technology will establish a new model for catalytic synergy. Synergy refers to the pre-organization of diverse enzymatic and binding elements (on a scaffolding molecule) in such a way that the resulting complex (‘catalytic complex’) achieves rate enhancements beyond what can be realized by simply mixing the corresponding free enzymes. The molecular basis of synergy is not well understood but it is considered to arise from a combination of binding domain-mediated targeting of the substrate to the catalytic complex (docking effect), and the spatial proximity of the different catalytic domains within the catalytic complex (proximity effect). Structural plasticity/flexibility has been proposed to be a key property of the scaffold that unifies these two phenomena and amplifies their effects. The technology is expected to offer numerous advantages.

Another unique aspect of the technology is that it produces functional biomaterials; i.e. various compositions of matter that retain (and enhance) the biological functions of the constituent proteins. This technology will offer many potential uses.

According to a first aspect, a first chimeric protein, wherein an amino acid sequence of a first protein is inserted into an amino acid sequence of a surface loop of a second protein, wherein the amino acid sequence of the first protein splits the second protein into an amino-terminal segment (N-segment) and a carboxy-terminal segment (C-segment), and further wherein the N-segment and the C-segment from the same chimeric protein are kept from assembling by the amino acid sequence of the first protein.

According to a second aspect, the chimeric protein further comprises a second chimeric protein comprising substantially the same sequence as the first chimeric protein, wherein the N-segment of the first chimeric protein assembles with the C-segment of the second chimeric protein. According to an embodiment, the C-segment of the first chimeric protein assembles with the N-segment of the second chimeric protein. According to an embodiment assembly may occur in response to a trigger.

According to a third aspect, the N-to-C terminal length of the amino acid sequence of the first protein is at least twice as long as the distance between C-alpha atoms of the two amino acids that define the termini of the surface loop of the second protein.

According to a fourth aspect, the amino acid sequence of the first protein comprises a thermodynamic stability equal to or greater than the thermodynamic stability of the amino acid sequence of the second protein.

According to a fifth aspect, in instances where triggered assembly is desired, the thermodynamic stability of the amino acid sequence of the first protein in the presence of the trigger is equal to or greater than the thermodynamic stability of the amino acid sequence of the second protein.

According to a sixth aspect, the amino acid sequence of the first protein is modified to adjust the thermodynamic stability of the amino acid sequence of the first protein, such that the thermodynamic stability of the amino acid sequence of the first protein in the presence of the trigger is equal to or greater than the thermodynamic stability of the amino acid sequence of the second protein.

According to another aspect, the first protein is Apoptosis Stimulating Protein of p53 2 (“ASPP2”) or an isoform of ASPP2, and the second protein is ubiquitin. The amino acid sequence of ASPP2 can be inserted into ubiquitin at amino acid position 63 or into ubiquitin at the amino acid position 19. Further, the first protein can be a ubiquitin-like protein possessing an amino acid sequence and N-to-C terminal distance similar to that of ASPP2. Alternatively, the first protein can be ubiquitin, and the second protein can be barnase, or the first protein can be GCN4, and the second protein can be barnase. The technology is also demonstrated using ubiquitin as the first protein and ribose binding protein (RBP) as the second protein. Combinations thereof are also possible.

According to another aspect, when the second protein is barnase, the N-segment is selected from the group consisting of amino acids: 1-22, 1-36, 1-47, 1-66, 1-79, or 1-103. When the second protein is RBP, the N-segment is selected from the group consisting of amino acids: 1-35, 1-60, 1-126, 1-211, or 1-259. When the second protein is ubiquitin, the N-segment is selected from the group consisting of amino acids: 1-19, 1-36, or 1-63.

According to another aspect, an isolated nucleic acid that encodes a chimeric protein, wherein an amino acid sequence of a first protein is inserted into an amino acid sequence of a surface loop of a second protein, wherein the amino acid sequence of the first protein splits the second protein into an N-segment and a C-segment, and further wherein the N-segment and the C-segment are kept from assembling by the amino acid sequence of the first protein.

According to another aspect, a polymer comprising at least two of a chimeric protein, wherein an amino acid sequence of a first protein is inserted into an amino acid sequence of a surface loop of a second protein, wherein the amino acid sequence of the first protein splits the second protein into an N-segment and a C-segment, and further wherein the N-segment and the C-segment are kept from assembling by the amino acid sequence of the first protein.

According to another aspect, the polymer comprises a hydrogel, the hydrogel comprising many thousands of chimeric proteins that are cross-linked to each other by the assembly of the N-segments with C-terminal segments in the manner described above.

According to another aspect, a method of creating a chimeric protein, wherein an amino acid sequence of a first protein is inserted into an amino acid sequence of a surface loop of a second protein, wherein the amino acid sequence of the first protein splits the second protein into an N-segment and a C-segment, and further wherein the N-segment and the C-segment are kept from assembling by the amino acid sequence of the first protein.

According to another aspect, the DSMs at one end of the target protein can be of one type and the DSMs on the other end of the target protein are of a different type. In yet another embodiment, all DSMs are comprised of a portion of an assembler protein linked by the lever protein to a portion of a different assembler protein.

According to another aspect, one or more of the DSMs of a chimeric protein construct comprise, for example, one or more of the following portions of Ubiquitin: Ub 1-19, Ub 20-76, Ub 1-36, Ub 37-76, Ub 1-63, or Ub 64-76, or one or more of the following portions of Barnase: Bn 1-22, Bn 23-110, Bn 1-36, Bn 37-110, Bn, 1-47, Bn 48-110, Bn 1-66, Bn 67-110, Bn 1-79, Bn 80-110, Bn 1-103, or Bn 104-110, or one or more of the following portions of Ribose Binding Protein: RBP 1-35, RBP 36-277, RBP 1-60, RBP 61-277, RBP 1-126, RBP 127-277, RBP 1-211, RBP 212-277, RBP 1-259, or RBP 260-277, among many other portions.

According to another aspect, a polymer comprising two or more chimeric proteins, wherein the chimeric proteins assemble randomly or, alternatively, form structures including, but not limited to, ring structures and ribbons among many other structures.

According to another aspect, DSMs appended to the termini of target proteins result in the proteins spontaneously assembling into soluble oligomers, viscous networks, or hydrogels, among other oligomers.

According to another aspect, one or more of the DSMs of a chimeric protein construct comprise, for example, a lever protein (such as ubiquitin) inserted into RBP, a 277-amino acid member of the periplasmic binding domain family of nonenzymatic receptors. According to one embodiment, the insertion points are one or more of amino acids 35, 60, 126, 211, and 259, although other insertion points might be possible.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic overview of the design of domain-swapping modules according to an embodiment.

FIG. 2 is a schematic overview of the mechanism of DSM-induced self assembly according to an embodiment.

FIG. 3 is a schematic of DSM constructs and X-ray structure of domain-swapped polymer according to an embodiment.

FIG. 4 is a ribbon diagram of Bn with destabilized Ub mutant (V26G) placed into each of the six surface loops and turns of Bn (centered at positions 22, 36, 47, 66, 79, and 103), according to an embodiment.

FIG. 5 is a ribbon diagram of BU103, with residues 1-103 and 104-110 of Bn extending from the N- and C-termini of Ub, respectively (FIG. 5A), according to an embodiment.

FIG. 6 is an alignment of the Bn and Ub domains of BU103 with their respective WT proteins, according to an embodiment.

FIG. 7 is a schematic of MEF design illustrated by ten existing constructs, according to an embodiment.

FIG. 8 is a schematic of MEF-induced domain swap, including the results of certain biochemical tests according to an embodiment.

FIG. 9 is a graph of the results of SEC chromatograms revealing oligomerization up to ˜14-mers (50 μM monomer).

FIG. 10 is a graph of the results of thermal denaturation indicating that RU35 is folded, stable, and able to bind ribose.

FIG. 11 is a schematic representation of various DSM protein constructs formed using target proteins 1 and 2, and assembler proteins A, B, C and E. A1, B1, C1 and E1 are the first part of assembler protein A, B, C and E respectively, and A2, B2, C2 and E2 are the remainder of the respective assembler protein A (e.g., A1=Ub 1-63, A2=Ub 64-76).

FIG. 12 is a possible structure that can result from mixing the DSM protein constructs (PCs) 1, 2, 3, 5, 6, and 7, and the floating DSM 12, according to an embodiment.

FIG. 13 is two examples of complex structures that can form even when a single DSM protein construct is used with the same DSM on both sides of the target protein (A-B-Targer-A-B).

FIG. 14 depicts four different DSM protein constructs, A-B-1-A-B, A-B-1-C-D, C-D-2-C-D, and A-B-2-C-D, where A and B are the complementary parts of a first assembler protein (e.g., Bn 1-66 and Bn 67-110), and C and D are the complementary parts of a second assembler protein (e.g., Ub1-19 and Ub 20-76). FIG. 14 also depicts that a mixture of these four DSM protein constructs can result in homo-oligomers, and that mixed oligomers are also possible.

FIG. 15 is a schematic representation of three unique DSM protein constructs. #1: X1-X2-Target 1-A1-C2; #2: Y1-Y2-Target.

FIG. 16 is a schematic representation of four unique DSM protein constructs that can be used to build a four DSM protein construct junction, each with one unique junction DSM.

FIG. 17 is a schematic representation of domain swapping by two single-DSM protein constructs, where the target protein of one is Beta-glucosidase and the target protein of the other is a CBM.

### DETAILED DESCRIPTION

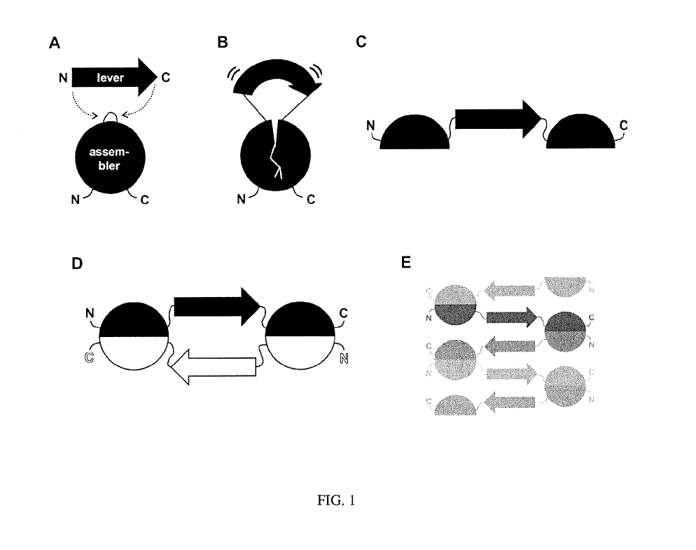

Referring now to the drawings, wherein like reference numerals refer to like parts throughout, there is seen in FIG. 1 an overview of the design of domain-swapping modules. Domain-swapping modules (DSMs) can be constructed from two proteins, hereafter called a ‘lever’ protein and an ‘assembler’ protein. A DSM is a fusion protein in which the lever is inserted into a surface loop of the assembler (FIG. 1A). The lever, which has a long amino (N) to carboxy (C) terminal distance, stretches the assembler (FIG. 1B) and splits it into two fragments, the N-fragment and the C-fragment (FIG. 1C). The assembler fragments are held apart by the lever so that they cannot rejoin. However, if the assembler protein is split at the proper location, the fragments will recombine with their respective counterparts from either one (FIG. 1D) or more (FIG. 1E) different but similarly-split assembler proteins. This type of recombination, which involves exchanging reciprocal segments of the polypeptide chain, is known as a domain swap. Domain swapping regenerates the original, pre-split structure as well as the original biological function of the assembler. The lever protein prevents the two fragments of a single DSM from binding to each other, but if two of the same DSMs encounter one another, the two assembler fragments of each DSM will domain swap with their complements (i.e., their other halves) from the other.

The purpose of DSMs is to cause the molecules to which they are fused to spontaneously self-assemble into one of several compositions of matter. According to another embodiment is provided methods to construct a set of highly efficient DSMs built by fusing the human protein ASPP2 (acting as lever) to the human protein ubiquitin (Ub, acting as the assembler). The assembler and lever proteins can be wild-type proteins, modified proteins, or specifically-engineered proteins. The DSMs constructed: (1) are small (14 kDa), (2) express at high levels in Escherichia coli (≧100 mg purified protein per liter), and are therefore inexpensive to produce, and (3) domain swap extremely efficiently, so that only oligomers are observed at 1 micromolar monomer concentration. According to another embodiment is the demonstration of the technology by the construction and testing of several examples, and various structures. In the present application various DSMs are designated, unless otherwise indicated or clear from the context, by the capitalized first letter of the lever protein followed immediately by the capitalized first letter of the assembler protein which is followed immediately by the amino acid position at which the lever protein is inserted into the assembler protein. For example, the DSM constructed by inserting ASPP2 into ubiquitin at amino acid position 63 is designated UA63, and in one embodiment the UA63 DSM is the amino acid chain (Ub1-63)-ASPP2-(Ub64-76). Other examples are BU36 for a DSM created by inserting ubiquitin into barnase at amino acid position 36, and RU126 for a DSM created by inserting ubiquitin into ribose binding protein at amino acid position 126. The letters may refer to wild type or modified versions of the proteins.

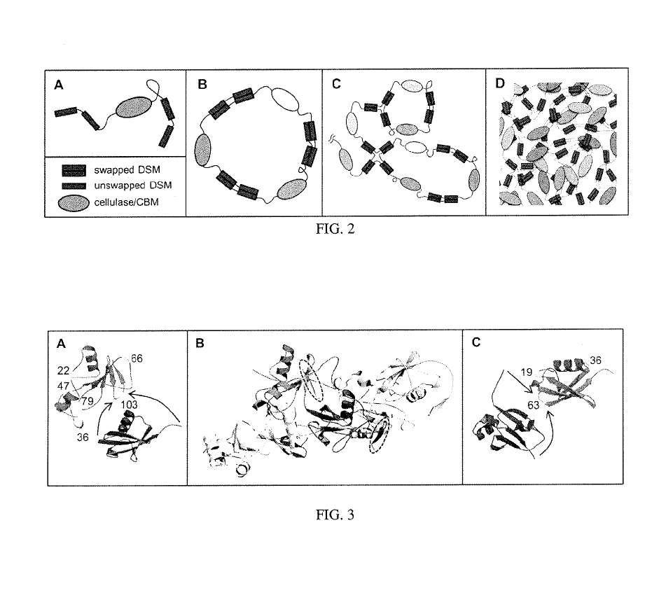

FIG. 2 illustrates the mechanism of DSM-induced self assembly. In this example, the target proteins (ovals) are cellulases and carbohydrate binding domains (CBMs), but can be other proteins. As monomer concentration is raised the material is predicted to consist of mainly: (A) monomers, (B) small, looped oligomers, (C) large, mixed looped-branched networks, (D) infinitely branched hydrogels. The target protein is the protein whose activity will provide function to the resulting material in one embodiment of the invention. In FIG. 2, the target proteins are cellulases and CBMs. One DSM is fused to each terminus of a target protein forming a DSM-target-DSM protein construct (DTDpc or DTD protein constructs), although constructs with only one DSM fused to a target protein are useful (target-DSM protein or TDpc or TD protein constructs; the terms “protein construct” and “DSM protein construct” are used to refer to both DTDpcs and TDpcs). A flexible peptide linker, generally consisting of 5-30 amino acids, is used to fuse the DSMs to the target protein so that the structure of the DSM will not interfere with the structure of the target protein and vice versa. End-to-end fusions such as this are common in many molecular biology protocols and it is generally expected, as experiments have demonstrated, that properly designed and constructed DSMs will not perturb the structure or function of target proteins. This construction allows the DTDpc to spontaneously organize into one of several compositions of matter, depending on initial monomer concentration. DSMs generate branched junctions when each of the two assembler proteins that make up the DSM self assemble with a complementary assembler protein that is part of a different DSM fused to another target protein. This capability is critical, as branching generates the cross-links that are required for formation of 3D meshes and hydrogels.

Once a DSM or DTDpc or TDpc is designed, a plasmid cassette can be constructed that will express the protein construct or DSM. Described herein is a plasmid cassette to enable rapid introduction of DSMs to any target protein, using standard molecular biology protocols. The DSM-target-DSM protein is then ready for bacterial expression and purification via the His-tag included in the cassette.

In simplified protein constructs, the DSM consists of both parts of a particular assembler protein separated by a lever protein, and all DSMs are of the same composition. In more complex protein constructs, different DSMs are used and fused to the same target proteins or to different target proteins or both. For example, the DSMs on one end of a particular target protein can be of one type while that on the other end can be another. In even more complex protein complexes, some or all DSMs may consist of one part of one assembler protein linked by the lever protein to one part of a different assembler protein. Appropriate construction of the DSMs and the protein constructs makes possible highly controlled rational design of complex protein structures.

### Example 1

Methods of DSM generation according to one embodiment. A DSM can be constructed at the genetic level, by, for example, ligating the gene encoding the lever protein into the gene encoding the assembler protein. The DSM protein is obtained by expressing the recombinant gene in a bacterial or eukaryotic cell expression system. A design specification of a DSM is that the lever protein must have an N-to-C terminal length at least twice as long as the distance between C-alpha atoms of the two amino acids that define the termini of the surface loop (into which the lever will be inserted) in the assembler protein. For simple applications (i.e. for materials that self-assemble without additional factors), the lever should have a thermodynamic stability comparable to or greater than that of the assembler, so that the lever forces the assembler to unfold and not vice versa. For applications where self-assembly is triggered by external perturbants (e.g. small molecules, pH/temperature change, etc.), the stability of the lever in the presence of perturbant should be comparable to or greater than the stability of the assembler. The lever protein can be a wild-type protein, a modified protein, or a specifically-engineered protein. For example, point mutations can be introduced into the lever, if necessary, to make it less stable than the assembler in the absence of perturbant. Ubiquitin (FIG. 3A, B), ASPP2 (FIG. 3C) and GCN4 (not shown) have been successfully employed as levers. The assembler can in principle be any protein with the exception of those that contain a disulfide bond that would physically prevent the domain swap. The assembler could similarly be a wild-type protein, a modified protein, or a specifically-engineered protein. The lever can be inserted into any surface loop of the assembler, and has been done so for the DSMs consisting of ubiquitin as lever and barnase as assembler (FIG. 3A) and ASPP2 as the lever and ubiquitin as the assembler (FIG. 3C). At the current time, the sites that will cause the assembler to domain swap must be experimentally determined. X-ray structure of the DSM in which the ubiquitin lever was inserted at position 103 of the barnase assembler (FIG. 3B) proves that the molecule self-assembles by the expected DSM mechanism.

An improved DSM design was created by inserting ASPP2 (lever) into ubiquitin (assembler) at position 63 of ubiquitin (FIG. 3C). Other engineered/composite lever/assembler structures have been created and many more can be created. The ASPP2/ubiquitin DSM is ready for commercial use using the plasmid expression cassette described above.

In one demonstration, GFP was chosen as the target protein and fused the DSM in FIG. 3C to each end. The DSM-GFP-DSM fusion protein was characterized as well as the DSM-DSM empty cassette. Results showed that at low concentration, the empty cassette self-assembles into soluble oligomers similar to that in FIG. 2B. Indeed, at high concentration, DSM-GFP-DSM forms a hydrogel. The gel is fluorescent, indicating that the biological activity of GFP is retained during the self-assembly process. Two additional constructs have been created where the same DSM was fused to barnase and staphylococcal nuclease. Both constructs show enzymatic activity comparable to that of the wild-type enzyme controls.

### Example 2

Methods for generating polymers, networks, and hydrogels from DTD protein constructs. DTD protein constructs were expressed in Escherichia coli following standard protocols for T7-based overexpression. DTD protein constructs were purified as follows. Bacterial cells were lysed and centrifuged (DTDpc is expressed as insoluble bodies that likely form as a result of self-assembly inside the cells). The supernatant was discarded and cell pellets were washed alternately with buffer followed by 0.5 M NaCl, repeating 3-5 times. The pellets were then dissolved in 8 M urea. At this point the DTD protein constructs are approximately 80% pure. The final purification step consists of binding the protein to either a nickel-NTA column (for His-tagged DTD protein constructs), or an ion-exchange column (for DTD constructs that do not contain a His-tag), followed by elution with imidazole or NaCl, respectively. The pure DTD protein constructs were then dialyzed against water or buffer to remove the urea. Polymers, networks, and hydrogels were prepared by concentrating the above material to the desired protein concentration using ultrafiltration, and allowing polymerization to proceed to completion.

### Example 3

As described above, domain swapping is a mechanism for forming protein dimers and oligomers with high specificity. It is distinct from other forms of oligomerization in that the binding interface is formed by reciprocal exchange of polypeptide segments. Swapping plays a physiological role in protein-protein recognition and it can also potentially be exploited as a mechanism for controlled self assembly. Our experiments have demonstrated that domain-swapped interfaces can be engineered by inserting one protein into a surface loop of another protein. The key to facilitating a domain swap is to destabilize the protein when it is monomeric but not when it is oligomeric. This condition is achieved by employing the ‘mutually exclusive folding’ design to apply conformational stress to the monomeric state. Ubiquitin is inserted into one of six surface loops of barnase. The 38 Å amino-to-carboxy terminal distance of ubiquitin stresses the barnase monomer, causing it to split at the point of insertion. The 2.2 Å X-ray structure of one insertion variant reveals that strain is relieved by intermolecular folding with an identically-unfolded barnase domain, resulting in a domain-swapped polymer. All six constructs oligomerize suggesting that inserting ubiquitin into each surface loop of barnase results in a similar domain-swapping event. Binding affinity can be tuned by varying the length of the peptide linkers used to join the two proteins, which modulates the extent of stress. Engineered, swapped proteins have the potential to be used to fabricate ‘smart’ biomaterials, or as binding modules from which to assemble heterologous, multi-subunit protein complexes.

Below are described further experiments which demonstrate that domain swapping can be induced by inserting one small protein (e.g., ubiquitin, 76 amino acids) into surface loops of another small protein (e.g., barnase, 110 amino acids). Any protein seems capable of undergoing a domain swap, but few actually do (there are ˜60 examples in the Protein Data Bank). Logically, the key to facilitating a domain swap is to destabilize the protein when it is monomeric but not if it is oligomeric. This unusual condition is achieved by employing the ‘mutually exclusive folding’ design to apply selective stress to the monomeric state, as illustrated schematically in FIG. 1. Ub was previously inserted into a surface loop of Bn at position 66 to generate barnase-ubiquitin 66 (BU66; FIG. 1A). Ub (38 Å N-to-C terminal distance) stretches Bn at the point of insertion while Bn simultaneously compresses the ends of Ub (FIG. 1B). This antagonistic interaction is parameterized by a coupling free energy which depends primarily on the length of the peptides used to link the two proteins. Very long linkers (10 Gly each) decouple the molecular tug-of-war and the coupling free energy is zero. If the linkers are sufficiently short (<2 amino acids), then the coupling free energy exceeds the folding free energy (AG) of the Ub or Bn domain and the more stable protein is predicted to unfold the less stable protein (FIG. 1C). This relationship can be represented by a thermodynamic box consisting nominally of four states [Bn(unfolded)-Ub(unfolded), Bn(folded)-Ub unfolded), Bn(unfolded)-Ub(folded), and Bn(folded)-Ub(folded)] linked by coupling and folding free energy terms.

Enzymatic, thermodynamic, and CD structural results as well as molecular dynamics simulations, however, called the four-state model into question. Those data suggested that inserting Ub (or the GCN4 DNA binding domain) into position 66 of Bn unfolded the latter, but Bn was able to bind to and refold with another copy (or copies) of itself to regenerate the active enzyme (FIG. 1D and FIG. 1E).

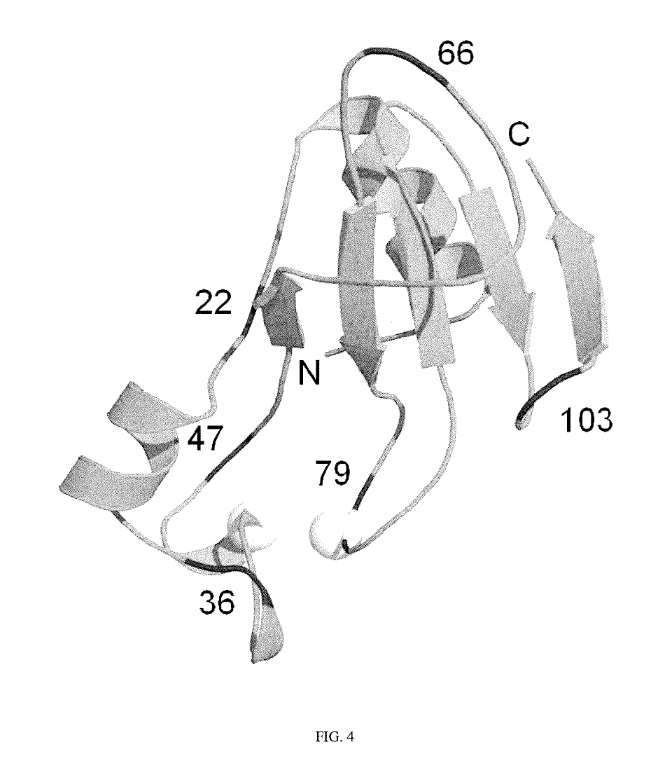

The conformational stress model predicts that inserting an unstructured or unstable protein into Bn will not dramatically destabilize Bn and thus not induce domain swapping. Accordingly, a destabilized Ub mutant (V26G) was placed into each of the six surface loops and turns of Bn (centered at positions 22, 36, 47, 66, 79, and 103; see FIG. 4 for locations and nomenclature), to test whether Bn can tolerate insertion while remaining folded and principally monomeric. With a ΔG value of 1.8 kcal/mol, V26G Ub was expected to exert relatively little stress on the more stable Bn domain (ΔG=11.5 kcal/mol for WT Bn; stability of the Bn domain is lower in the BU variants due to loop-closure entropy loss). TABLE 1 summarizes stability parameters measured by Trp fluorescence. The Ub domain does not contain any Trp residues so fluorescence reports only on the conformation of the Bn domain. The Bn domain was destabilized, as anticipated, but it remained folded in all six constructs (Table 1). BU66 (V26G) is the most stable. One possible reason is that the 66-loop, being the largest loop in Bn, can accommodate insertion with the least perturbation to flanking Bn residues. Size exclusion chromatography finds that all V26G variants are predominantly monomeric, although minor peaks corresponding to oligomeric states up to tetramers are observed. Bn therefore seems robust in its ability to accept unstable or unfolded proteins into its surface loops while remaining folded.

TABLE 1
Stability parameters of the Bn domain of BU variants (where Cₘ
is the midpoint of urea-induced denaturation, protein concentration
is 5 μM (monomer), and errors are standard deviations of three
measurements).

| Variant | Cₘ (M) | ΔG (kcal/mol) | m (kcal/mol/M) |
|---|---|---|---|
| BU22 (V26G) | 1.27 ± 0.08 | 3.19 ± 0.80 | 2.49 ± 0.56 |
| BU36 (V26G) | 2.79 ± 0.02 | 7.83 ± 0.23 | 2.80 ± 0.06 |
| BU47 (V26G) | 1.70 ± 0.01 | 4.42 ± 0.13 | 2.60 ± 0.10 |
| BU66 (V26G) | 3.42 ± 0.02 | 9.54 ± 1.25 | 2.81 ± 0.37 |
| BU79 (V26G) | 2.75 ± 0.06 | 7.31 ± 0.07 | 2.65 ± 0.07 |
| BU103 (V26G) | 3.1 ± 0.04 | 8.64 ± 0.64 | 2.79 ± 0.22 |
| BU22 | 0.22 ± 0.04 | 0.54 ± 0.07 | 2.43 ± 0.24 |
| BU36 | 1.66 ± 0.03 | 3.42 ± 0.28 | 2.05 ± 0.15 |
| BU47 | 0.63 ± 0.06 | 1.06 ± 0.12 | 1.69 ± 0.16 |
| BU66 | 2.70 ± 0.07 | 4.30 ± 0.30 | 1.59 ± 0.16 |
| BU79 | 1.69 ± 0.10 | 3.68 ± 0.42 | 2.13 ± 0.36 |
| BU103 | 2.62 ± 0.03 | 5.45 ± 0.98 | 2.09 ± 0.38 |

The investigation next intensified the conformational stress exerted by Ub and determined whether Bn unfolds or oligomerizes. Disruptive force was generated by replacing V26G Ub with WT Ub (ΔG=6.9 kcal/mol) at the same insertion points. In agreement with the thermodynamic model, stabilizing the Ub domain destabilizes the Bn domain by an additional 2.7-5.2 kcal/mol (average ΔΔG=−3.8±0.9 kcal/mol; TABLE 1). Still, the Bn domain remains folded and stable in all variants except for BU22. For BU22, the midpoint of denaturation (Cₘ) is 0.22 M urea and ˜20% of the protein is unfolded in the absence of denaturant. Size exclusion chromatography, however, reveals that the constructs now exist predominantly as dimers, trimers, and tetramers. The distribution of oligomeric states varies among mutants. BU103 is mainly tetrameric with a smaller population of dimer. BU22 is almost exclusively dimeric, and BU36, BU47, BU66, and BU79 exist as a mixture of trimers, dimers, and monomers. Because of the multiplicity of oligomeric states, the observed folding transitions are likely not two state (although all curves fit satisfactorily to the two-state linear extrapolation equation (not shown)). Free energies reported in TABLE 1 are therefore apparent values that depend on protein concentration (5 μM in the present experiments).

Disulfide cross linking was employed to ask whether the BU66 dimers observed above associate via domain swapping. The Bn double-cysteine mutant (A43C+S80C) was used, which is known to readily form an intramolecular disulfide bond (FIG. 4). This bond cross links the two fragments of Bn that the model predicts to be split in BU66. If BU66 dimers bind in a conventional manner, i.e. via complementary patches on the monomer surface, then the C43-C80 disulfide bond is expected to form intramolecularly and the dimers will dissociate to monomers on reductant-free SDS-PAGE. If BU66 dimers are domain swapped, then C43 of one monomer is predicted to form an intermolecular S—S bridge with C80 of the second monomer (and vice versa), thereby crosslinking the black and white semicircles in FIG. 1D. This species will migrate on the gel as a covalent dimer. BU66 (A43C+S80C) was denatured and reduced in guanidine hydrochloride/dithiothreitol then allowed to oligomerize and oxidize in the absence of denaturant and reductant. SDS-PAGE shows that the dimer peak recovered from size exclusion chromatography is the intermolecularly cross-linked dimer, whereas the monomer peak is the intramolecularly cross-linked monomer. This result suggests that BU66 (A43C+S80C) monomers bind via a domain swap, yielding the closed, symmetrical dimer depicted in FIG. 1D.

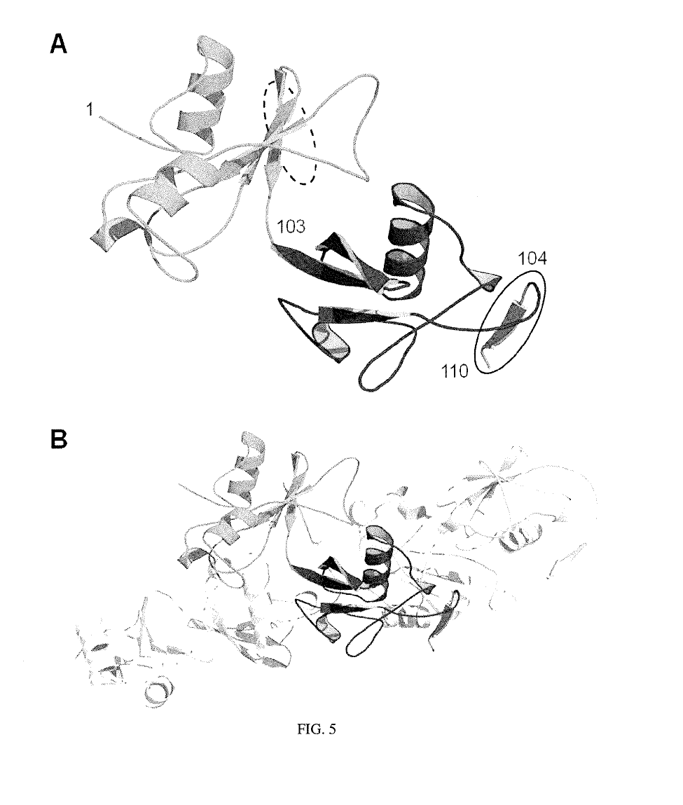

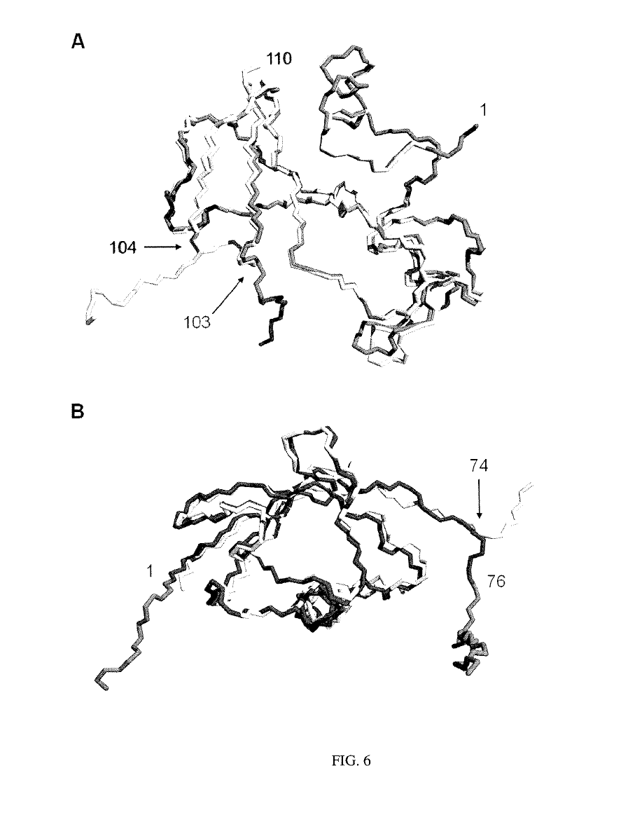

To determine the structural basis for oligomerization, we solved the X-ray structure of BU103 to 2.2 Å resolution. The dimeric species above was isolated and concentrated to 1.4 mM for crystallization. As a result of the increased concentration, the dimers spontaneously reorganized to form long, linear polymers (FIG. 1E). The asymmetric unit, however, consists of a single BU103 monomer (FIG. 5A). In WT Bn, residues 104-110 form the last strand of the five-stranded β-sheet (FIG. 4). The structure of BU103 shows that Ub has pulled apart Bn, with residues 1-103 and 104-110 of Bn extending from the N- and C-termini of Ub, respectively (FIG. 5A). The binding interface is clearly revealed as a domain swap. The 1-103 and 104-110 fragments of Bn do not contact each other within the same molecule; they are separated by more than 30 Å. Rather, residues 104-110 insert into the BU103 molecule in the next asymmetric unit, replacing the missing fifth β-strand (FIG. 5B). Similarly, the absent fifth strand of the central BU103 molecule in FIG. 4B is supplied by the 104-110 fragment from the monomer in the preceding asymmetric unit. These asymmetric units are related by successive application of the crystallographic three-fold screw operator, generating a helical polymer extending the length of the crystal. While the extent of oligomerization and regular helix observed in the crystal are enforced by high concentration and crystal packing, it seems reasonable to assume that the oligomers seen in solution at lower concentration are connected in the same way. Oligomerization thereby restores native interactions in the Bn domain without conformational stress.

Comparing the Bn domain of BU103 with WT Bn finds the structures of inter- and intramolecularly folded Bn to be very similar. Amino acids 3-102 of WT Bn can be superimposed on the same residues of the Bn domain of BU103 with a C_{α} root mean-square deviation (RMSD) of 0.57 Å (FIG. 6A). Superposing just that subset of amino acids causes Bn residues 104-109 (from the preceding BU103 molecule) to align on the corresponding residues from WT Bn with a maximum C_{α} RMSD of 0.94 Å at Q104, and an overall C_{α} RMSD of 0.66 Å. The swapped β-strand adopts virtually the same structure as the fifth strand in WT Bn, as evidenced by the all-atom RMSD value of 0.75 Å for residues 105-108. BU103 thus satisfies all three criteria for a classically domain-swapped oligomer.

Like the Bn domain of BU103, the structure of the Ub domain is nearly identical to that of its WT counterpart. Amino acids 1-70 of WT Ub superimpose on the corresponding residues of the Ub domain of BU103 with an all-atom RMSD of 0.47 Å (FIG. 6B). Significant differences are limited to the C-termini. Beginning at R74 and continuing to the end of Ub at G76, the direction of the polypeptide backbone diverges in the two molecules. This result indicates that the C-terminus of Ub is flexible. Some flexibility at one or both of Ub-Bn junction points may be desirable in order to accommodate any rigid-body adjustments that may need to be made at the binding interface.

The above results suggest that it is possible to engineer a self-assembly mechanism based on mutually exclusive folding-induced domain swapping. The general approach is to insert a ‘lever’ protein into a surface loop of an ‘assembler’ protein. As long as the N-to-C and loop-termini distances of the lever and assembler exceed the aforementioned ratio, the global free energy minimum of the system (above a threshold protein concentration) is an oligomer in which the lever and assembler are both folded with the latter protein domain swapped (FIG. 1E). This mechanism for self-assembly is distinct from those developed by others (e.g. coiled-coils, naturally-occurring repeat proteins) in two respects. First, our mechanism is based on conformational stress. In that respect it is reminiscent of the naturally-occurring polymerization reaction of the serine protease inhibitor α₁-antitrypsin. The native fold of α₁-antitrypsin is metastable. Proteolytic cleavage of the inhibitor's reactive center loop by the target protease permits the cleaved loop to adopt a β-strand conformation and insert into the central β-sheet of the same α₁-antitrypsin molecule, thereby allowing it to attain its global free energy minimum. In a spontaneous process that is aided by several disease-causing mutations, the reactive center loop can mis-insert into another α₁-antitrypsin molecule, triggering a daisy chain reaction that generates highly stable fibers. The basis for the metastable nature of monomeric α₁-antitrypsin is not well understood. In contrast, mutually exclusive folding-induced conformational stress—and thus the affinity of the binding reaction—can be controlled by known physical and thermodynamic principles. One straightforward method is to vary the length of the linkers that join lever to assembler. It has previously been shown that very long linkers fully decouple the mutually exclusive folding interaction between the Ub and Bn domains. Linkers of ten Gly each result in the BU66 monomer being fully relaxed; no oligomerization is detected. Shortening the linkers one residue at a time gradually increases stress and causes the dissociation constant (K_{d}) for dimerization to decrease. When Ub and Bn are fused without any additional linkers, BU66 dimerizes with sub-micromolar K_{d} (K_{d} cannot be determined accurately due to the presence of higher-order oligomers).

Another method for tuning binding affinity is to modulate thermodynamic stability of the lever protein. It is demonstrated above that a more stable Ub domain is better able to unfold Bn and induce it to oligomerize. Since protein stability is coupled to ligand binding as well as to solution conditions, the domain-swapped binding interaction can in principle be switched on and off by the presence of ligands or changes in temperature, pH, or salt concentration. It is important to recognize that, since neither of the above two modes of affinity tuning modifies the binding interface, they are not expected to compromise the inherent specificity of the domain-swapped interaction. Indeed, it is the combination of high specificity and moderate affinity that likely explains why nature chose domain swapping to mediate cell-cell interactions.

The second distinction of the present design is that it may be modular. There is no reason to expect that Bn is unique among potential assemblers in its ability to domain swap upon forced unfolding. This view is supported by the observation that Bn responds in the same way—by forming oligomeric complexes—as a result of being pulled apart at different locations. This is not the case, however, for all mutually exclusive folding constructs. A mutually exclusive folding chimera was previously constructed by another group in which the 27^{th} Ig domain of titin was inserted into a surface loop of the GB5 protein. That group was able to directly observe folding of the Ig domain and concomitant unfolding of the GB5 domain in real time. Subsequent refolding/domain swapping of GB5 was not detected. As to the lever, any stable protein with a moderately long N-to-C distance should be able to perform the same stretching function as Ub. Additional lever-assembler combinations are needed to test the generality of the domain swapping mechanism.

The structures of both domains in the domain-swapped oligomer of BU103 are virtually identical to those of WT Ub and WT Bn. This finding, together with gel filtration and disulfide crosslinking results from the other BU insertion variants, means that it is feasible to create self-assembling oligomers and polymers that retain and integrate the functions of the parent molecules. This novel mechanism allows for precise control of both the structural details of protein-protein binding interfaces as well as the strength of their interaction

Materials and Methods

BU genes were created by inserting the Ub gene into the Bn gene at the positions indicated. The genes were fused using nucleotides encoding Gly-Gly and Gly as the first and second linkers, respectively, and the Ub gene lacked the N-terminal Met. BU proteins were purified as described. Urea denaturation studies were carried out in 10 mM sodium phosphate (pH 7.0), 0.1 M NaCl, 10° C. Unfolding of the Bn domain was monitored by Trp fluorescence (Ub does not contain any Trp residues), and unfolding data were fit to the two-state linear extrapolation equation ΔG(urea)=ΔG−m[urea] as described. Oligomerization experiments were performed by denaturing BU variants in 2 M guanidine hydrochloride, then refolding the samples by rapid 20-fold dilution to a final protein concentration of 20 μM. Samples were then injected onto a Superdex-75 gel filtration column (GE Healthcare). Disulfide crosslinking experiments were performed as above except 10 mM dithiothreitol was present during denaturation. After dilution, the samples were dialyzed to remove reductant and allowed to oligomerize under oxidizing conditions prior to injection onto the Superdex-75 column. BU103 was crystallized at 20° C. using the hanging-drop vapor-diffusion method with the mother liquor consisting of 10 mM Tris (pH 8.0), 1 M (NH₄)₂SO₄, 1.5% isopropanol (v/v). X-ray diffraction data were collected at station A1 at the Macromolecular Diffraction Facility at the Cornell High Energy Synchrotron Source (MacCHESS), reduced using HKL-2000, phased by molecular replacement (1UBQ and residues 3-103 of chain A of 1A2P), and refined using Phenix. X-ray statistics are listed in TABLE 2. The coordinates for BU103 have been deposited with PDB accession code 3Q3F.

TABLE 2
X-ray data collection and refinement statistics.

| Beam line | CHESS-A1 | |
|---|---|---|
| Number of crystals | 1 | |
| Space group | P 3₂21 | |
| Unit cell (Å) | a = 86.872, b = 86.872, c = 75.571 | |
| Angle (°) | α = 90, β = 90, γ = 120 | |
| Resolution range (Å) | 37.7-2.17 | (2.23-2.17)^{a} |
| R_{merge} | 0.151 | (0.505) |
| I/σ_{I} | 12.9 | (2.39) |
| F/σ_{F} cutoff | 0 | |
| # Reflections | 16849 | (1106) |
| Completeness (%) | 94.5 | (85.0) |
| R_{cryst} | 0.1899 | |
| R_{work} | 0.1853 | (0.2333) |
| R_{free} | 0.2316 | (0.2927) |

| | |
|---|---|
| Free R value test set size | 9.98%, 1681 (124) |
| Wilson B factor | 22.66 |
| # Heavy atoms refined | 1792 |
| # Solvent atoms | 230 |
| Estimated coordinate error (Å) | 0.27 |
| RMSD bond | 0.007 |
| RMSD angle | 1.008 |
| RMSD chirality | 0.079 |
| RMSD dihedral | 13.389 |

### Example 4

Methods for creating self-assembling, bioactive polymers, networks, and hydrogels that are capable of interacting with and reacting to their environment. This interaction elicits a two-way response that can either alter the properties of the material (e.g. for biosensing purposes) or induce a change in the surrounding environment. This pursues the latter capability with the specific goal of overcoming the longstanding hurdle that has limited the feasibility of converting plant cellulose to biofuels: enzymatic hydrolysis of cellulose to glucose. The problem is twofold. First, many proteins and enzymes are needed to break down cellulose, including binding proteins, exocellulases, and endocellulases. Second, these enzymes—organized into bodies called cellulosomes in certain microorganisms—work synergistically in cells, but natural as well as engineered cellulosomes have met with only limited success at the scale of industrial cellulose processing. Simply put, most of the enzymatic players are known but not the mechanisms by which they can be made work together synergistically outside of the cell.

Described is a solution to both problems that is created using a protein engineering method that employs 3D domain swapping to generate self-assembling polymers that can be linear or extensively branched. The degree of branching can be controlled to yield materials whose form ranges from soluble to viscous to soft, moldable hydrogels. Self assembly is achieved via protein-in-protein fusion constructs that we pioneered and have subsequently minimized to create highly efficient DSMs that are appended to the termini of an enzyme of choice. The enzymes and binding proteins are incorporated into the growing polymer, and data indicate that they retain their native structures and catalytic activities in the final network.

The resulting material is expected to offer the following unique advantages over existing artificial cellulosomes. (i) Existing cellulosomes are discrete ‘hard’ particles and this limits the extent and duration of their interaction with cellulose polymers. The proposed cellulosomes are viscous or soft materials in which cellulases and binding domains are integrated in flexible structures ranging in size from small oligomers to effectively infinite three-dimensional networks. These networks can envelop the polymeric substrate using many thousands of binding domains, bringing to bear an even greater number of diverse hydrolytic domains. (ii) Existing cellulosomes typically array a limited number of enzymes on a relatively small scaffold. By contrast, there is no limit to the number or type of enzymes and binding domains that can be incorporated into our domain-swapped networks, and it is straightforward to create compositions with defined stoichiometries simply by mixing the starting subunits in the desired ratio. Different DSMs can be used to further organize the components into functionally optimal patterns.

Biological deconstruction of cellulose currently consists of three steps: (i) pretreatment of raw lignocellulose (often with chemicals and heat) to forms that are more amenable to biodegradation, (ii) enzymatic hydrolysis of the pretreated material to glucose, and (iii) microbial fermentation of glucose to ethanol. Enzymatic hydrolysis has traditionally been the efficiency-limiting step and the proposed work directly attacks this longstanding problem.

Cellulose is a semi-crystalline, insoluble substance that resists breakdown. Biological hydrolysis requires the concerted action of a group of proteins including endocellulases (both processive and nonprocessive), exocellulases, and carbohydrate binding modules (CBMs). In an effort to enhance hydrolytic performance, researchers have attempted to increase the stabilities of the individual proteins as well as to improve the total activity of the mixture. Significant progress has been made towards the former goal but not towards the latter.

In some cellulolytic fungi and bacteria, cellulases and CBMs are arranged in extracellular structures called cellulosomes. These bodies consist of a proteinaceous scaffold (scaffoldin) to which up to ˜100 cellulases/CBMs can be attached. Natural cellulosomes digest cellulose efficiently because, due to the preorganization of binding and catalytic components, they achieve rate enhancements beyond what can be realized by simply mixing the corresponding free proteins. We refer to this property as positional synergy. The molecular basis of positional synergy is not well understood but it is considered to arise from a combination of CBM-mediated targeting of the cellulosome to the substrate (docking effect), and spatial proximity of the different hydrolytic domains (proximity effect). Structural plasticity has been proposed to be a key property of the cellulosome that unifies these two phenomena. Progress toward recapitulating synergy with artificial cellulosomes has been incremental and this remains a primary goal.

Described herein are methods for creating self-assembling cellulosomes that are designed to enhance the properties suspected to give rise to positional synergy to unprecedented levels. Previously pioneered is ‘mutually exclusive folding’ (MEF), a mechanism by which the folding free energy of one protein is used to unfold a second protein into which the first was inserted. Here MEFs are employed to generate ‘domain-swap modules’ (DSMs). DSMs are small proteins that as described above, when appended to the termini of target proteins, cause the tagged proteins to spontaneously assemble into soluble oligomers, viscous networks, or hydrogels in the absence of any other factors. Importantly, biological activities of the target proteins are retained in the final compositions. This technology makes it possible to establish the optimal set of proteins to create the most active complexes and to determine the ideal size, stoichiometry, and viscoelastic properties of the formulations to maximize hydrolysis of real biomass substrates.

The materials that can be created are predicted to offer the following specific advantages. (i) Diversity. Unlike existing cellulosomes, there is no limit to the number or type of enzymes and CBMs that can be incorporated into the material. Stoichiometry is adjusted by mixing the starting subunits in the desired ratio. Different DSMs, each of which can only swap with an identical DSM, can be employed to further organize the components into predetermined patterns. (ii) Plasticity. Conventional cellulosomes are discrete ‘hard’ particles. In contrast, the bioactive proteins here are arrayed in flexible structures ranging from oligomers to essentially infinite 3D meshes. These networks allow the material to bind the cellulose polymer with many thousands of CBMs, bringing to bear an even greater number of hydrolytic domains. The flexibility, density, and porosity of the network can be controlled in order to optimize synergy between functional subunits as well as the physical properties of the material. (iii) Economy and scalability. The amount of bioactive gels and networks that can be generated is limited only by the amount of protein that can be synthesized. Current DSMs express well in bacteria and available data indicate that they can improve expression of the target protein to which they are fused. For example, we currently purify DSM-tagged green fluorescent protein (GFP) from E. coli with yields in excess of 100 mg/L starting culture (compared to ˜10 mg/L for free GFP). This trend, if borne out by the constructs outlined in this proposal, suggests that very large quantities of DSM-tagged cellulases can be produced easily and cheaply by scaling up bacterial growths using large bioreactors.

Choice of Cellulosome Proteins.

According to one embodiment, cellulosomes are assembled from the four protein types that are minimally required to completely hydrolyze cellulose to glucose: a CBM, an endocellulase, an exocellulase, and a β-glucosidase for the final step. Each type is represented by multiple families of related proteins so the number of permutations is combinatorial. One the main advantages of the proposed material is that, unlike discrete scaffold particles, the different permutations can all be incorporated into the proposed material so that every combination can be present within the resulting large protein networks. Moreover, the position of each protein relative to others can be specified so proteins that work best in close proximity to each other can be close, or adjacent, and those that work best at a distance can be distant from each other. In one embodiment the cellulosomes comprise four proteins, a CBM, an endocellulase, an exocellulase, and a β-glucosidase, in an interlinked network, linked to form a chain or linked into a looped oligomer. In one embodiment, the cellulosomes comprise one or more variations of a CBM, one or more variations of an endocellulase, one or more variations of an exocellulase, and one or more variations of a β-glucosidase that are in an interlinked network, linked to form a chain or linked into a looped oligamer.

CBMs can exist as domains of secreted free cellulases (in aerobic microorganisms) or as independent proteins attached to the cellulosome scaffold (in anaerobic microorganisms). The E7 and E8 proteins from the CBM33 family appear to be important for binding crystalline cellulose and possibly disrupting its structure and in one embodiment they will be incorporated into the DSM protein construct. Other CBMs recognize additional forms of cellulose, e.g. single and multiple polymers, and these will be tested alone and in combination with E7/E8.

It makes sense to use enzymes from the aerobic bacterium Thermobifida fusca because these proteins are particularly well characterized, highly active, thermostable (T. fusca grows at ˜50° C.), and have been cloned and expressed in E. coli BL21. The similarly well-studied enzymes from the bacterium Cellulomonas firmi and the fungi Trichoderma reesei are attractive secondary targets. Some combinations of T. fusca endocellulases (5A, 6A) and exocellulases (6B, 48A), when attached pairwise to a scaffold, exhibited modest (≦2-fold) increases in specific activity toward crystalline cellulose. All possible mixtures of these same enzymes are incorporated in materials with the addition of 9A endocellulase. 9A is processive whereas 5A and 6A are not. Processivity is an important property of natural cellulosomes. Dense, heavily branched networks present the greatest number of catalytic domains for hydrolyzing the polymer at multiple sites, but mobility of each protein within the network is expected to be limited. Smaller, less branched networks may offer fewer but more mobile catalytic domains. Since beta-glucosidase acts on soluble sugar substrates, it is not clear whether this enzyme will be more effective as a component of a cellulosome or as a free enzyme, and both are worth trying. In one embodiment, the cellulosomes comprise one or more variations of a CBM, one or more variations of an endocellulase, and one or more variations of an exocellulase that are in an interlinked network, linked to form a chain or linked into a looped oligamer, and these cellulosomes are added with beta-glucosidase to a cellulose containing liquid. It should be noted that many of the above enzymes are inhibited by their products (but most of them are not inhibited by glucose). The capability of making hydrogels is expected to address this potential problem by enabling solid materials (such as beads) to be coated with cellulolytic hydrogels, thus allowing the cellulose to be processed in a continuous, rather that batchwise, process.

Domain-Swap Modules for Self Assembly.

DSMs are generated by inserting a protein (the lever) into a surface loop of another protein (the assembler), such that the N- to C-terminal distance of the lever is greater than the distance between the ends of the surface loop in the assembler. This insertion is done at the genetic level, by ligating the gene encoding the lever into the gene encoding the assembler, at the position that corresponds to the desired surface loop of the translated assembler protein. Ten DSMs have been created: six in which the lever is ubiquitin (Ub) and the assembler is barnase (Bn), one in which the lever is GCN4 and the assembler is Bn, and three in which the lever is the Ub-like protein ASPP2 and the assembler is Ub (FIG. 1A-C)(other Ub-like proteins may be able to be used to create additional DSMs). In addition, another group made an MEF construct that employs the small proteins 127 and GL5 as the lever and assembler. In each case, a molecular tug-of-war ensues in which the lever (being the more stable of the two proteins) rips apart the assembler at the point of insertion. However, it is thermodynamically unfavorable for the assembler to remain unfolded, and above a characteristic concentration (K_{d}) it binds to and refolds with other identically-unfolded assemblers to generate domain-swapped oligomers and long polymers. For this study the Ub-ASPP2 DSM was exclusively employed because it swaps very efficiently (K_{d}<100 nM; see below). The 2.2 Å crystal structure of one of the Bn-Ub DSMs (Ub inserted into position 103 of Bn) confirmed that the subunits bind via a specific domain-swapped binding interface (FIG. 1D). It is expected that each insertion variant of a given DSM will generate a unique interface such that only identical DSMs will swap with each other.

Crosslinking is necessary to generate networks and gels. Crosslinking is achieved by fusing a DSM to each terminus of a target protein, causing the material to exhibit protein concentration-dependent branching (FIG. 2). At concentrations below K_{d} the lowest energy state of the protein is a monomer in which the DSMs are unfolded (FIG. 2A). At concentrations that are low but above K_{d}, the most stable structures are those in which each DSM swaps with only one partner. This results in closed, circular oligomers such as the trimer in FIG. 2B. These species are small and fully soluble. As the concentration is increased it becomes increasingly likely that a DSM will swap with two different DSMs, generating a branch point. FIG. 2C depicts a large network that contains a mixture of closed loops (dimer and trimer) joined by branched junctions. In this state the composition is expected to be of highly variable size and viscosity. At high concentrations the material becomes essentially infinitely branched (FIG. 2D). It does not crystallize; rather, it forms a soft, highly hydrated substance known as a hydrogel.

To test the predictions in FIG. 2, a plasmid expression cassette was created that contains two concatenated Ub-ASPP2 DSM genes separated by a restriction site into which a gene of interest can be inserted. The GFP gene was inserted and the DSM-GFP-DSM fusion protein exclusively employ as well as the DSM-DSM empty cassette. At 1 micromolar monomer concentration, electron microscopy reveals that the empty cassette forms soluble oligomers of roughly 12-24 protomers (FIG. 3A). At ≧500 micromolar monomer concentration DSM-GFP-DSM forms a hydrogel (FIG. 3B). Lower concentrations resulted in viscous solutions that did not gel, consistent with structures in FIG. 2C. The fluorescence of the gel indicates that GFP retains its native structure in the matrix. As expected for a true hydrogel, the material does not dissolve when placed into aqueous solution. Hydrogels may address the issue of product inhibition because they can potentially be coated onto immobilized surfaces to facilitate continuous-mode processing (rather than batch mode), so that substrates can be replenished and products can be removed continuously.

Characterization of Compositions.

There are many possible formulations. The activity of each formulation can be tested toward pharmaceutical-grade microcrystalline cellulose, by monitoring the appearance of reducing ends as well as free glucose. It is also of value to characterize the mechanical and network properties of formulations that perform significantly better than controls (i.e. equivalent mixtures of free enzymes) so that better formulations can be devised. Techniques useful for characterization include dynamic frequency sweeps to measure torsional shear, compression assays, thermogravimetric analysis to measure water content of gels, and stability measurements in realistic solution conditions. Once formulations that work well under more idealized conditions, their activity against more complex substrates (e.g. kraft wood pulp which contains hemicelluloses and lignin in addition to cellulose) can be evaluated with the ultimate goal of hydrolyzing real-world pretreated biomass. If necessary, hemicellulases and hemicellulose binding modules can be designed into to the DSM protein constructs.

### Example 5

Methods and systems for constructing and using DSM constructs for medical purposes. It is known that hydrogels can be used as delivery vehicles. Hydrogels are natural or synthetic polymers that can be made to mimic the extracellular matrix. They are hydrophilic, highly hydrated and usually crosslinked to enable them to take on soft, moldable 3D shapes. Hydrogels have garnered interest in recent years because they can encapsulate a wide range of substances—from small molecules to proteins to living cells—in a stable, biologically compatible, and biodegradable network. The main applications for hydrogels include drug delivery, tissue engineering, and 3D cell culture.

Developers of traditional hydrogels are faced with a number of daunting challenges. The first is to make the material biologically compatible. The substances used to make the majority of existing hydrogels were selected for their safety to humans and for their ability to gel, not for their biological function. Indeed, in keeping with their purpose of delivering drugs, many of these substances (e.g. polyethylene glycol (PEG), oligosaccharides, and short, self-assembling peptides) are intended to be inert although this is not always the case. PEG, for example, is not readily degradable and it engenders an immune response that includes antibody production. The second challenge is to encapsulate the cargo with high yield. The third is to control the rate of release, which is often limited by (rapid) diffusion. Slower, zero-order kinetics are usually the goal.

By leveraging the rich functionalities and biocompatibility of proteins, using 3D domain swapping to assemble the protein polymers can be used to design gels that largely bypass the above hurdles. A domain swap is defined as reciprocal exchange of polypeptide segments between two or more monomers, with the structures of the oligomer and monomer being identical except at the points of strand exchange. This interaction can produce closed ring-shaped oligomers or polymers of indefinite length. An example of the former is demonstrated by the cadherin family of proteins, which mediate cell-cell adhesion and development of tissue architecture. The latter includes runaway swapping as observed in serpinopathies and prion amyloidoses, in which domain-swapped fibrils are associated with cytotoxicity. There is no risk that our molecules will induce fibrillization in humans since only the engineered proteins are capable of swapping. Recent work has shown that several proteins can be induced to form swapped polymers, including cystatin C, GB1, and RNase A. However, general principles have not yet emerged and these remain isolated cases. The hydrogels engineered according to one embodiment stand apart from others in at least the following key respects for example:

Functionality.

The polymers are comprised of functional proteins; they are not simply inert networks for encapsulating cargo. Moreover, because each subunit in the polymer (DSM protein construct) contains one molecule of the target protein, the specific activity of our gel is expected to be significantly higher than that of a conventional gel in which the target protein carrying capacity of the gel is likely to be comparatively less. Specific activity can be dialed down if necessary. The target protein or proteins are selected based upon the desired biological activity the gel is intended to have.

Generality.

Preliminary results suggest that the mechanism by which monomeric proteins are induced to self-assemble into polymers is general. Generality means that hydrogels can in principle be created from combinations of different lever and assembler proteins, or from a single lever/assembler pair joined in different ways. This versatility increases the chance of generating gels that retain the functions of the parent proteins as well as possess the requisite mechanical properties.

Controlled Release.

Since the DSM protein construct gels do not encapsulate cargo, the traditional concept of release rate does not apply. The duration of the biological response is instead expected to be proportional to the rate at which the gel breaks down. A straightforward procedure for controlling the extent of polymerization can be accomplished by modulating the affinity of the domain swapping interaction, providing a means for tuning the functional lifetime of the gels to meet any desired application. According to one embodiment, a DSM protein construct provides multiple means for tuning the breakdown of the gel. For example, the DSMs used to create the DSM protein constructs in the gel can be designed to breakdown predictably over time, under variable conditions (pH, temperature, electromagnetic conditions, salinity, etc.), or in the presence of a chemical or biological agent. To facilitate this tuning, the lever protein and/or the assembler protein can be modified. For example, the terminal ends or ends of the assembler protein can be modified to breakdown in a known manner under certain conditions.

Localization.

A general advantage of hydrogels is that, once introduced into the body, they persist at the site of administration until they are broken down. This property is especially important for gels containing growth factors and cytokines since their systemic application would likely risk serious side effects. The gels are naturally biodegradable (e.g. by endogenous enzymes) unlike some synthetic polymers. They also have the additional attribute that only the polymer is functional. Any freely-circulating monomers generated by breakdown or left over from incomplete gelation will be inactive.

Biocompatibility.

The DSM protein construct gels can be designed for maximum biocompatibility. The exclusive use of human proteins in their construction minimizes potential cytotoxicity and immugenicity.

Creating Functional Hydrogels from Domain-Swapped Target Protein.

Polymers can be created using our mutually exclusive folding approach. Mechanical and rheological properties of the gels can be measured and then optimized by: (i) inserting Ub into different surface loops of each protein, (ii) varying the length of the peptides used to fuse lever to assembler, and (iii) changing the protein concentration at which the material is formed. The advantage of the present methods is that, since the lever and assembler proteins retain their native structures and biological properties, the hydrogel can itself possess biological activity independent of and in addition to that of the encapsulated cargo (if any). For example, the lever and/or assembler protein can possess the function of binding to a specific cell-surface receptor, which would target the encapsulated cargo to that specific cell type.

A preferred ‘domain-swapping module’ (DSM) consists of Ub as the assembler and the Ub-like protein ASPP2 as the lever. This DSM is small, very soluble, and swaps extremely efficiently. One DSM is placed at each of the N- and C-termini of the target protein via short peptide linkers to make the fusion DSM-target protein-DSM protein construct. Each DSM can swap with two different molecules, generating branch points at both ends. In this way, a highly crosslinked network of DSM protein constructs is formed. The advantages of this design are that no chemical crosslinking reagents are used and the target protein has the highest probability of remaining functional in gel form. It is neither assembler nor lever and it is modified in a simple, non-perturbing way.

Once a DSM protein construct gel is made, it can be tested for cytotoxicity and for its stability toward degradation and hydrolysis under physiological conditions. Protein secretion and gene expression can be profiled in each cell-gel construct, to establish whether physiological signaling is taking place. Changes in cell viability and morphology will be characterized by microscopy and confocal imaging. Mechanical stability of the gels will be investigated in the context of biomechanical stimulation of the cells. Finally, appropriate cells can be assayed for markers indicative of the biological activity the gel is desired to have, in order to gauge the biological potency of the gels.

Results—Mutually Exclusive Folding

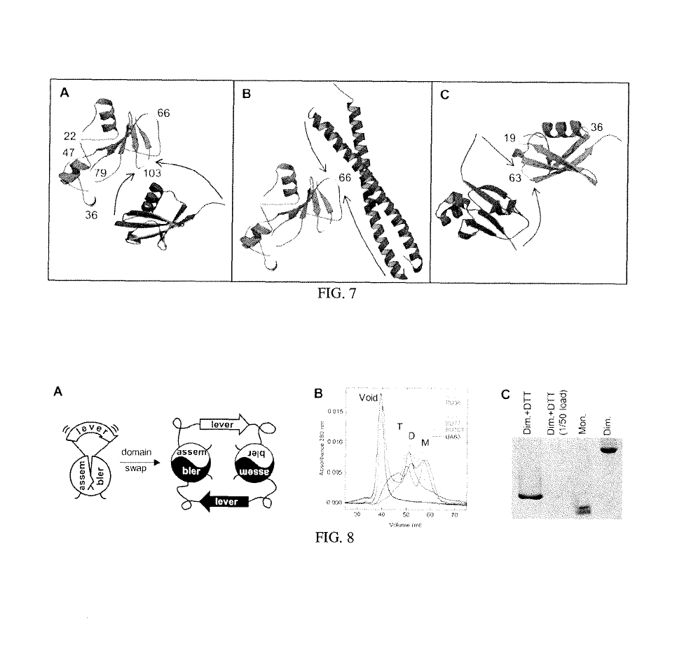

Hydrogels are created using the protein engineering approach that was previously developed, named ‘mutually exclusive folding’ (MEF). MEF entails inserting a lever protein into a surface loop of an assembler protein. Ten such chimeras/DSMs have been created as described above. In addition, another group made an MEF construct that employs the small proteins 127 and GL5 as the lever and assembler. In each case, if the N- to C-terminal distance of the lever (40 Å for Ub) is greater than the distance between the ends of the surface loop of the assembler (typically ≦10 Å), a tug-of-war ensues in which the lever stretches the assembler at the point of insertion, and the assembler simultaneously compresses the lever. A formalism was developed that parameterizes this antagonistic interaction using a coupling free energy that depends primarily on the lengths of the peptides used to link the two proteins. If the linkers are sufficiently short, then the coupling free energy exceeds the folding free energies of either protein and, in theory, the more thermodynamically stable protein unfolds the less stable protein. This prediction holds true when protein concentration is below a threshold value. However, for all constructs in FIG. 7 an unusual refolding event was observed at higher concentrations and this phenomenon forms the basis of our hydrogel design.

For each of the proteins in FIG. 7 the lever is predicted to rip apart the assembler at the point of insertion. It was therefore surprising that the Bn domains of BU66 and BG66 retain RNase activity and that the Ub and GCN4 domains appear to be folded as well. It was hypothesized that this seeming paradox is resolved by intermolecular folding between assembler domains in the form of a domain swap (FIG. 8A). Superdex-75 size exclusion chromatography (FIG. 8B) indicates that all six BU insertion variants oligomerize at 10 μM concentration, suggesting that domain swapping of the assembler may be a general response to internal insertion of the lever.

To test whether oligomerization occurs via swapping, the Bn double-Cys mutant (A43C+S80C) was employed, which is known to readily form an intramolecular disulfide bond. This bond cross links the two fragments of Bn that would be split by Ub in BU66. If BU66 dimers interact in a conventional manner, i.e. via complementary patches on the monomer surfaces, the C43-C80 disulfide bond will form intramolecularly and the dimers will dissociate to oxidized monomers on reductant-free SDS-PAGE. If BU66 dimers are domain swapped, then C43 of one monomer will form an intermolecular cross link with C80 of the second monomer (and vice versa), resulting in a species that migrates on the gel as a covalent dimer. Purified, oxidized BU66 (A43C+S80C) dimer and monomer run as interlinked dimer and intralinked monomer, respectively, on SDS-PAGE (FIG. 3C). This result strongly suggests that BU66 dimers form via domain swapping.

The X-ray structure of BU103 was solved to 2.2 Å resolution. In WT Bn, residues 104-110 from the last strand of the five-stranded β-sheet (FIG. 7A). Ub has pulled apart Bn, with residues 1-103 and 104-110 of Bn extending from the N- and C-termini of Ub, respectively. The structure clearly shows that these fragments have swapped with those of the adjacent protomers, generating a linear, helical polymer that extends the length of the crystal. The structures of the Bn and Ub domains of BU103 superimpose on those of the corresponding free proteins with all-atom RMSDs of less than 1.0 Å, consistent with our functional studies which suggested that fusion and subsequent oligomerization does not perturb native structure of either lever or assembler. Oligomerization thereby restores native interactions in the Bn domain while relieving conformational strain that was induced by Ub insertion.

To further explore the generality of the polymerization mechanism, small, ubiquitin-like protein ASPP2 was employed as the lever and Ub was transferred to the role of assembler. ASPP2 was inserted at position 63 of Ub to create UA63. FIG. 8B reveals that UA63 oligomerizes even more efficiently than the BU variants. At a monomer concentration of 10 μM, UA63 elutes as a single peak in the void volume of the Superdex 75 column, suggesting and apparent molecular weight of >150 kDa. Unlike BU, no monomeric or low-order oligomeric species are observed. Circular dichroism (CD) spectra (not shown) indicate that UA63 is folded. Transmission electron microscopy (TEM) shows that, at 1.0 μM monomer concentration, UA63 appears to form oligomers and linear polymers of up to ˜100 nM in length. This length corresponds to roughly 12-24 protomers (2×10⁵−4×10⁵ Da), depending on how they are arrayed in the polymer. It is likely that the polymers are considerably longer at the protein concentrations employed in the gelation experiments described below (2 mM monomer).

It is important to note that the Ub fusion proteins will probably not be substrates for the cellular E1/E2/E3 pathway because these enzymes need a free C-terminus on Ub. Nor will the molecules likely be targeted by proteasomes, as recognition requires a string of least four Ub's linked via K48. In the unlikely event that the molecules interfere with cellular Ub pathways, K48 and other reactive Lys such as K63 can be changed to Arg. As the gel breaks down, any monomers of the assembler protein that may arise and circulate in the body will be nonfunctional. Self-inactivation helps confine biological activity to the site of application.

Having pioneered the MEF technique and made dozens of MEF chimeras, it is possible to suggest the following guidelines for determining which proteins can be induced to swap and oligomerize. (i) The assembler should be relatively stable as an independent protein (ΔG_{fold}≧4 kcal/mol or so) to overcome the entropic penalty associated with intermolecular folding. The stability of the lever should be comparable to or greater than that of the assembler. (ii) One should choose surface loops or turns in the assembler for inserting the lever. Beyond that, one cannot predict which sites will result in the most efficient swapping, however because there are typically a very limited number of surface loops in most proteins, the best site for inserting the lever protein can be readily found by testing each. (iii) The length of the peptides used to link lever to assembler is important and it determines the extents of swapping and polymerization. Fusing the lever and assembler with two ten-Gly linkers decouples the MEF effect and no oligomerization is observed. Oligomerization gradually increases with decreasing linker length, reaching a maximum with zero Gly. In addition to being thermodynamically stable, if large quantities of the DSM protein construct are desired, it is preferable that assembler should be expressed at reasonable levels in E. coli.

Advantageous criteria for a DSM is that it be small, soluble, stable, and most importantly, swap as efficiently as possible. Available data indicate that UA63 satisfies all four conditions. Results suggest that UA19 may be even better: it appears to be more thermally stable than UA63 (UA19 does not denature below 90° C.) and it can be concentrated to 9 mM without visible precipitation.

In one example, shown in FIG. 9, UA63 DSMs are fused to the N and C-termini of FGF2. One main consideration for linkers is that they not be so long that the DSMs can loop around and swap within the same molecule. If self-closure is a problem (as determined by predominantly monomeric species on gel filtration), then one can concatenate two or more target molecules to increase the distance between the termini of the fusion protein (e.g. DSM-FGF2-FGF2-DSM). In addition, gelation is preceded by crosslinking, which in turn is controlled by protein concentration as described below.

Gelation can be triggered by simply increasing protein concentration. No chemical crosslinkers need be added. At low protein concentration the molecules form mostly linear, unbranched polymers. The reason is that each DSM will likely swap with a single DSM from another subunit. Higher concentrations will drive each DSM to swap with DSMs from two other monomers to generate the gel. The main variables for controlling viscoelastic properties are length of linkers used to join lever to assembler and protein concentration. Both can be varied in order to optimize the mechanical and rheological properties of the gels.

It is important to be able to control the functional lifetime and specific activity of the gels because prolonged stimulation by some proteins can be deleterious to the cellular, intercellular and bodily processes. For example, this means that the gels may retain their signaling capability for only several days or for as long as perhaps several weeks. The rate at which the gel breaks down in the body depends on intrinsic properties of the gel as well as on extrinsic factors (mainly endogenous proteases). With regard to the latter, other investigators previously introduced MMP cleavage sequences into peptide-based gels in order to hasten their degradation in vivo. Through variation of the number of Gly residues that link lever to assembler, our design allows an additional and more straightforward mode of control. If gels are too long lasting, then one can increase the Gly linker length to decrease the affinity of the domain-swapped interaction, thus favoring disassembly. MMP recognition sequences can be introduced into surface loops and linker peptides as well, if necessary.

One can ‘dial down’ the specific activity of these gels by first making point mutants of the active protein so that it is unable to bind its receptor, then mixing them in various ratios with monomers containing working variations. The resulting gels may contain a mixture of functional and nonfunctional subunits, with overall specific activity being roughly proportional to their initial ratio.

It is also possible that activity of the gels may be too low. Loss of function would likely have one of two causes. The first is steric inhibition arising from interference from the Ub lever or from proximity to the DSMs. However, preliminary results with Bn indicate that insertion of Ub and polymerization does not appreciably perturb Bn structure or function. Nevertheless, some combinations of Ub insertion sites and Gly linker lengths minimize steric interference. Moreover, it is straightforward to increase the distance between the target protein and the DSMs by lengthening the tethering peptides although we must be alert to self-closure if the two DSMs attached to the target protein are complementary. Still, whether the monomer self-closes or crosslinks with other monomers reflects a local concentration-dependent equilibrium and one should be able to manipulate it by both linker length and protein concentration.

After a gel or polymer has been formulated for a particular purpose using the above methods and a particular set of active, linker and DSM proteins, a number of tests can be performed to assess its function and appropriateness to the particular application for which it was designed.

Degradation Testing and Protein Release.

A first step is to quantitatively analyze material degradation and hydrolytic susceptibility under simulated physiological conditions. A real-time degradation test can be performed on cylindrical constructs in PBS at 37° C. according to ISO 10993-13:2010 standards. Measures of degradation include mass balance, water uptake, and changes in the viscoelastic properties and network characteristics. In addition, release of the functional proteins with time into the PBS at 37° C. can be analyzed by enzyme-linked immunosorbant assay (ELISA). If the protein concentrations are below the detectable range for ELISA, single-plex assays for use with the Luminex platform can be used instead. Samples may be assayed at the following time points until mass loss stabilizes or structural integrity is lost: 0 h, 12 h, and then daily for two weeks, followed by every other day up to 3 weeks, and then every third day up to one month. Additional samples should be kept in reserve, should testing need to be performed beyond one month.

Cytotoxicity Analysis.

Each material developed should be screened using the direct contact assay, with one or both of the liquid extract or indirect contacts added in cases where results of the direct contact assay are inconclusive. Tissue culture polystyrene and sodium dodecyl sulfate can be used as negative (non-toxic) and positive (toxic) controls, respectively. The cells used can be L929 mouse fibroblasts. Cell viability in liquid extract and direct contact tests can be determined using the WST-1 metabolism and LIVE/DEAD viability assays. Cell viability in indirect contact tests can be quantified by manually scoring of neutral red stained samples. Each set of assay takes about 2-4 days, allowing rapid testing as materials are developed.

Cell Selection.

Target protein activity can be assayed by monitoring migration and proliferation of keratinocytes and fibroblasts.

Cell-Gel Construct Preparation.

The functional protein solution can be combined with cells just prior to the addition of the appropriate crosslinker. Appropriate cell-gel constructs can be cultured for 7-10 days. For all cell types, the control gels (run in parallel to the experimental samples) can be the PEO and Tisseel materials described above. The time scale can be chosen based on the time period required for the desired cellular activity (e.g., upregulation, down regulation) to develop in pellet culture and comparable 3D culture systems.

Viability and Morphology Analysis.

For viability and morphology, microscopy-based analyses of cell viability and morphology comparable to our prior work in 3-dimensional gels and 2-dimensional substrates can be performed. Cytoskeletal architecture and cell morphology can be visualized through staining of microfilaments (e.g., phalloidin staining of actin) and the plasma membrane (e.g., CellMask staining), respectively. Confocal imaging can be performed on samples cultured for 2, 4, and 7 days. Cell morphology can be quantified in terms of 3-dimensional cell orientation, elongation, and surface area to volume. LIVE/DEAD staining can be used to monitor cell viability.

Protein Secretion and Gene Expression Analysis.

To evaluate how contact with each of the functional hydrogels modulates cellular activity, assays can be performed to determine extracellular matrix and pro-/anti-inflammatory cytokine secretion. Following incubation of the cells with the functional hydrogel constructs for 2, 4, and 7 days, multiplex immunoassay on the Luminex platform can be used to monitor alterations in the cytokine profiles. Other aspects, such as matrix production, can be measured using approaches we have previously described including imaging, wet weighting, glycosaminoglycan and DNA assays, and histological and immunohistochemical analyses. Results can be compared to those obtained from cells grown in under other conditions. To assess gene expression, cell-gel constructs can be pulverized using cryogenic grinding with a Pellet Pestle (Kontes) and analyzed by quantitative PCR. Expression levels can be normalized against the most stable of ten reference genes (ACTB, B2M, GAPD, GUSB, HPRT1, PGK, PP1A, RPL13A, TBP and TFRC) as determined using a reference gene evaluation tool (www.leonxie.com/referencegene.php) that yields results from common reference gene finder programs, including Genorm, Normfinder, and Bestkeeper.

Mechanical Stability Testing and Mechanostransduction.

To investigate both the mechanical stability of cell-gel constructs and the extent to which biological activity of gels compares to and/or interacts with biomechanical stimulation of cells, constructs can be cultured with and without compressive mechanical stimulation between rigid plates in an FX-4000 Compression Plus system (Flexcell, Hillsborough, N.C.) using established protocols, with unloaded discs as the control. Based upon the known mechanosensitive nature of these cell types, changes are expected relative to unloaded cultures.

Materials created from DSM technology will establish a new model for catalytic synergy. Synergy refers to the pre-organization of diverse enzymatic and binding elements (on a scaffolding molecule) in such a way that the resulting complex (‘catalytic complex’) achieves rate enhancements beyond what can be realized by simply mixing the corresponding free enzymes. The molecular basis of synergy is not well understood but it is considered to arise from a combination of binding domain-mediated targeting of the substrate to the catalytic complex (docking effect), and the spatial proximity of the different catalytic domains within the catalytic complex (proximity effect). Structural plasticity/flexibility has been proposed to be a key property of the scaffold that unifies these two phenomena and amplifies their effects. The technology is expected to offer the following advantages:

Diversity.

Unlike conventional catalytic complexes, there is no limit to the number or type of catalytic and binding domains that can be incorporated into the material. Stoichiometry is adjusted by mixing the starting subunits in the desired ratio. Different DSMs, each of which can only swap with an identical DSM, can be employed to further organize the components into predetermined patterns.

Plasticity.

Conventional catalytic complexes are discrete ‘hard’ particles. In contrast, the bioactive proteins here are arrayed in flexible structures ranging from oligomers to essentially infinite 3D meshes. These networks allow the material to bind the substrates with multiple binding domains, bringing to bear an even greater number of catalytic domains. The flexibility, density, and porosity of the network can be controlled in order to optimize synergy between functional subunits as well as the physical properties of the material.

Economy and Scalability.

The amount of bioactive gels and networks that can be generated is limited only by the amount of protein that can be synthesized. Current DSMs express well in bacteria and available data indicate that they can improve expression of the target protein to which they are fused. For example, DSM-GFP-DSM (where GFP stands for green fluorescent protein) has been purified from E. coli with yields in excess of 100 mg/L starting culture (compared to ˜10 mg/L for free GFP). This trend suggests that very large quantities of catalytic another target protein. This capability is critical, as branching generates the cross-links that are required for formation of 3D meshes and hydrogels.

Ability to Control the Self-Assembly Process.

For many applications it is useful to trigger polymerization and/or gelation by an external perturbant such as addition of a small molecule, change in temperature, change in pH, exposure to light, etc. Appropriate choice of the lever protein allows for this type of triggering mechanism. Consider the example of initiating gelation by addition of a small molecule ligand. A protein that binds that ligand is selected for the lever. The natural thermodynamic linkage between ligand binding and protein stability dictates that addition of the ligand will stabilize the lever, thus imparting the lever with a greater ability to split apart the assembler. For polymerization to be initiated by addition of ligand, the lever must be less stable than the assembler in the absence of ligand (in which case the assembler stays intact and does not domain swap), and more stable than the assembler in the presence of ligand. The invention specifies how point mutations can be rationally introduced into the lever to meet this condition. In order to initiate self-assembly by the other examples mentioned above, one would choose a lever protein that becomes more thermodynamically stable upon shifting temperature, changing pH, or absorbing light (there are examples of all of these proteins in nature).

Applications.

The unique aspect of the technology is that it produces functional biomaterials; i.e. various compositions of matter that retain (and enhance) the biological functions of the constituent proteins. Examples of potential uses are as follows:

Biological Deconstruction of Cellulose to Glucose for Production of Ethanol and Other Liquid Biofuels.

Current efforts in the field to create artificial cellulosomes (combinations of cellulases and CBMs linked via chemical scaffolds) have failed to demonstrate synergy. The most efficient catalysts are expected to be large meshes or chains, as they likely will offer the best combination of structural flexibility and catalytic diversity.

Wound Healing/Regenerative Medicine.

Here, the target proteins are anti-inflammatory growth factors and/or cytokines. The material is assembled into hydrogels, which are implanted/injected at the site of the wound or at an arthritic joint. The invention specifies how the lifetime of the hydrogels can be controlled, once they are implanted.

Drug Delivery.

The simplest version of this application is to use DSM-generated hydrogels as inert matrices for encapsulating drugs. The material is implanted/injected as above, and the drug is slowly released over time as the gel breaks down. The more advanced version of this application is to assemble the hydrogels from proteins that bind to a cellular receptor, such that the drug is targeted to specific cells or organs.

Controlled Assembly and Disassembly.

Many hydrogel applications, such as the drug delivery application mentioned above, greatly benefit if: (1) gelation can be triggered by the addition of a small molecule, and (2) gelation can be reversed in a controlled manner so that the gel breaks down in the body. Chemical crosslinking reagents are currently used to initiate gelation in some commercial hydrogels, but these agents can be harmful, and they irreversibly crosslink the material. DSM technology potentially allows polymerization and crosslinking to be triggered by the reversible binding of a physiological cofactor (e.g. metal ions or vitamins) to a lever protein that naturally binds that cofactor, as described above. The gel then breaks down in the body as the cofactor diffuses out of the material and is metabolized normally.

### Example 6

To further test the generality of the DSM mechanism, and to identify the lever/assembler combination that will be most effective at creating self-assembling, catalytic biomaterials, an RU series of DSMs was created (RU35, RU60, RU126, RU211, and RU259). RUs are composed of Ub inserted into the indicated positions of ribose binding protein (RBP), a 277-amino acid member of the periplasmic binding domain family of nonenzymatic receptors. We explored the use of RBP as the assembler because RBP is extremely soluble (as well as stable). A limitation of the UA series of DSMs for certain uses is that, owing to their poor solubility, they may require dissolution in denaturant followed by lengthy concentration to generate soluble complexes and hydrogels.

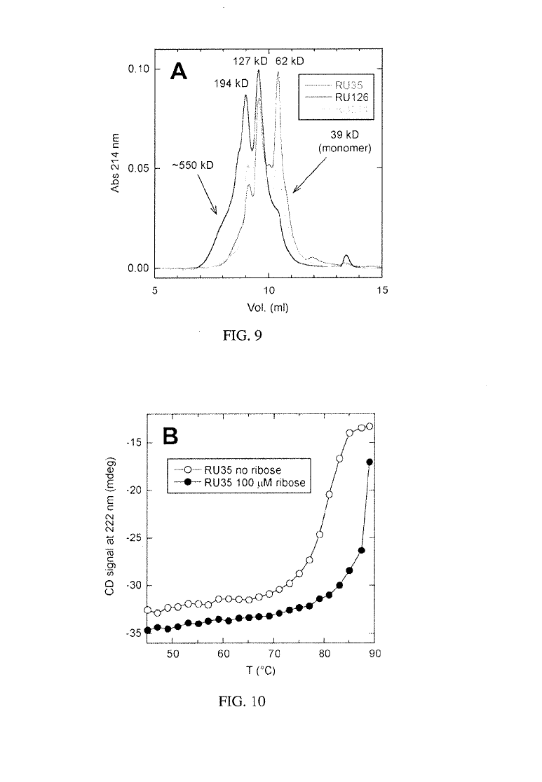

All five RU constructs expressed with very high yield in E. coli (>80 mg per liter of starting culture). It was possible to dissolve the freeze-dried, purified protein to 3-5 mM concentration in water without the addition of denaturant. This is approximately a 100-fold improvement over the UA series, which could only be dissolved at low micromolar concentration without first adding urea or other denaturant. To test whether the RU series self-assembles into complexes size exclusion chromatography (SEC) experiments were performed. All five constructs were found to form oligomers up to at least 5-mers, as shown in FIG. 10 for RU35, RU126, and RU211.

The next test was whether these oligomers form as a result of the predicted domain-swapping mechanism. To do so peptides consisting of eight glycines were used to link Ub to RBP. This group's previous study [Cutler et al. (2009) J. Mol. Biol 386, 854] found that the long, flexible glycine chains decouple conformational strain, thereby eliminating unfolding of the assembler and subsequent domain swapping. Preliminary SEC results with RU126 and RU259 indicate that the long linkers strongly reduce oligomerization (not shown). This experiment is the most definitive test of the DSM mechanism short of a high-resolution structure (which we are currently pursuing by X-ray crystallography).

Finally, to determine whether the RU series retains function in the self-assembled state, thermal denaturation studies were carried in the presence and absence of ribose, the natural substrate of RBP. The melting temperatures of all five constructs except for RU259 were raised by the addition of ribose, indicating that they possess wild-type binding activity. FIG. 10B shows the result for RU35, the least stable of the RU series.

In summary, the RU series of DSMs offers a significant advantage over the UA series. RU DSMs can be dissolved in seconds in water or buffer without the use of denaturants.

### Example 7

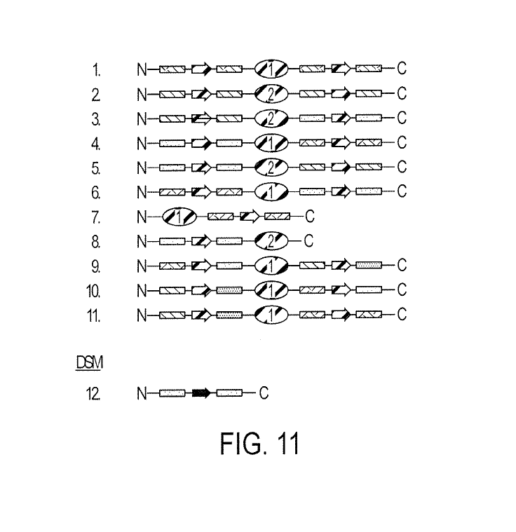

FIG. 11 shows various example DSM protein constructs formed using target proteins 1 and 2, and assembler proteins A, B, C and E. A1, B1, C1 and E1 are the first part of assembler protein A, B, C and E respectively, and A2, B2, C2 and E2 are the remainder of the respective assembler protein A (e.g., A1=Ub 1-63, A2=Ub 64-76). The arrow is the lever protein (e.g., ASPP2). The first and second parts of the assembler proteins will domain swap. Constructs 1-2 have the same DSM on both the N and C side of the target protein (B1-B2, or E1-E2). As a result, construct 1 will form a polymer only with other construct 1s, and construct 2 will form a polymer chain or loop only with other construct 2s. Constructs 3-6 consist of two different DSMs, one on each side of the target protein, although each of the DSMs consists of both parts of the same assembler protein. Construct 3 can join with constructs 4, 5 or 6 because all four incorporate the same DSM (A1-A2) on either the N or C side of their target protein. Construct 4's C end can only bind with the N end of Construct 6 or with another Construct 4 at its C end, since those are the only occurrences of the DSM B1-B2.

Constructs 7 and 10 are single DSM protein constructs. In Construct 7, its single DSM is attached to the C terminus of the target protein 1, while in Construct 8, its single DSM is fused to the N terminus of target protein 2. Single DSM protein constructs can be used to terminate a polymer chain of two DSM protein constructs, or as part of more complicated structures. The target protein is attached only at one end which may make it more accessible to chemicals in its environment and potentially more active.

Constructs 9, 10 and 11 are different from the other DSM protein constructs shown in that each of their DSMs consist of parts of two different assembler proteins. The DSMs of Construct 9 are B1-A2 on the N side of target protein 1 and E1-C2 on its C side. The DSMs of Construct 10 are E1-C2 on the N side of target protein 1 and B1-A2 on its C side. The DSMs of constructs 9 and 10 will not domain swap with each other because there are no complements to the assembler proteins that comprise them (i.e., there is E1, the first part of assembler protein E [e.g., Ub 1-63] but no E2, the second part of assembler protein E [e.g., Ub 64-76]). In Construct 11, the C side DSM consists of two B2s, the second part of assembler protein B, either of which may domain swap with the B1 part of one of the DSMs of either Construct 9 or 10 (or 1, 4, 6, or 7). Its other DSM is E2-C1. By mixing parts of different assembler proteins to make DSMs, the interactions of DSM protein constructs can be tightly controlled to build very specific structures.

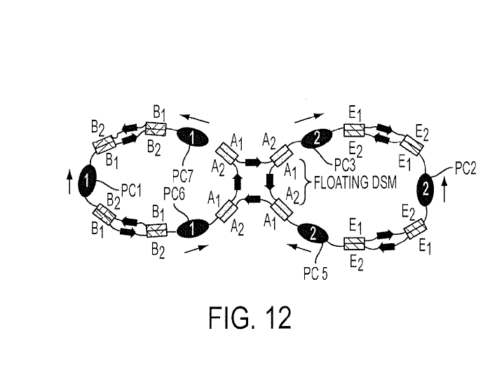

Number 12 in FIG. 11 is a DSM that is not fused to a target protein. It can be used to generate more complex structures. FIG. 12 shows a structure of which a ‘floating’ DSM a part. FIG. 12 shows one possible structure that can result from mixing the DSM protein constructs (PCs) 1, 2, 3, 5, 6, and 7, and the floating DSM 12.

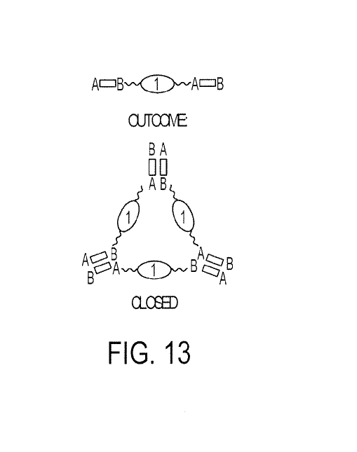

FIG. 13 gives two examples of complex structures that can form even when a single DSM protein construct is used with the same DSM on both sides of the target protein (A-B-Targer-A-B). Note that in FIG. 13, a different nomenclature is used than in FIGS. A and B. The DSM protein construct consists of a single DSM (A-B) on both sides of the target protein 1, which consists of A, the one half of an assembler protein, and B, the other half of the same assembler protein (i.e., A will domain swap with B). FIG. 13 shows a closed oligomer loop formed of three of the DSM protein constructs of FIG. 13. The DSMs on the ends of each protein construct bind to those on the end of another. A closed oligomer loop of any number of proteins can be formed with the single DTDpc shown in FIG. 13. FIG. 13 shows a more complex protein structure consisting of two joined closed loops of three proteins each, completely formed of the DTDpc show in FIG. 13. Each of the closed loops can contain any number of DSM protein constructs.

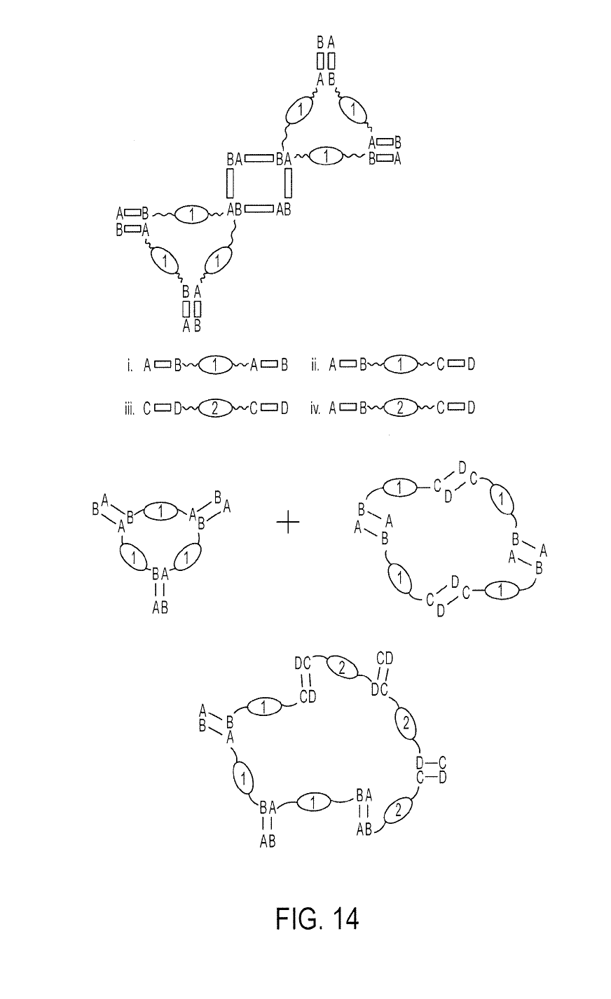

FIG. 14 shows four different DSM protein constructs, A-B-1-A-B, A-B-1-C-D, C-D-2-C-D, and A-B-2-C-D, where A and B are the complementary parts of a first assembler protein (e.g., Bn 1-66 and Bn 67-110), and C and D are the complementary parts of a second assembler protein (e.g., Ub1-19 and Ub 20-76 or Ub 1-36, Ub 37-76). FIG. 14 shows a mixture of these four DSM protein constructs can result in homo-oligomers, such as those in FIG. 13. FIG. 14 shows that mixed oligomers are also possible.

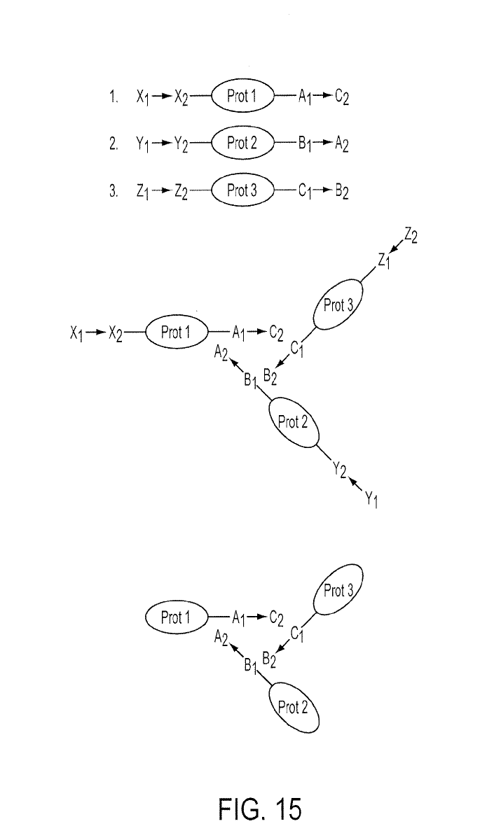

FIG. 15 shows three unique DSM protein constructs. #1: X₁-X₂-Target 1-A₁-C₂; #2: Y₁-Y₂-Target 2-B₁-A₂; and #3: Z₁-Z₂-Target 3-C₁-B₂. Custom junction DSMs on the C side of the target proteins are designed to interact in a very specific way to form the structure shown in FIG. 2E (they are referred to as junction DSMs only because they are intended to form a custom junction, not because of any underlying structural difference from other DSMs). One of the assembler protein parts in the custom junction DSM for DTDpc 1 can only domain swap with an assembler protein part in the custom junction DSM of DTDpc 2 (A₁ with A₂), and the other can only domain swap with an assembler protein part in the custom junction DSM of DTDpc3 (C₂ with C₁), and the remaining assembler protein parts of the custom junction DSMs of DTDpc 2 and 3 can only domain swap with each other (B₁ with B₂). These interactions can only form the three-protein construct junction at the center of the protein complex in FIG. 15. The DSMs on the other N end of the three DSM protein constructs must not interact or domain swap with the custom junction DSMs, otherwise the three protein constructs could combine into oligomeric complexes with other structures. In the example shown, these DSMs don't interact with each other, however in another embodiment, they can domain swap either fully or partially with each other, allowing direct connection of multiple protein complexes. In one embodiment, none of the three DSM protein constructs have N side DSMs, and they combine to form a complex having three dangling target proteins, as shown in FIG. 15. Unlike a three-protein-construct protein complex which has no free DSMs left to domain swap with a fourth protein construct, the complex three protein construct protein complex shown in FIG. 15 has three ‘free’ DSMs that can react with additional protein constructs (up to six if one of each of the three free DSMs domain swaps with only one of the assembler protein parts of another DSM protein construct).

One advantage of having DSMs constructed from assembler protein parts that do not interact with each other is the lever protein no longer has the requirement to force the two assembler protein parts apart, as it does when the two parts want to domain swap with each other. This means that the lever protein becomes more of a spacer protein, and can be long or short, soft or hard, depending upon the structural and chemical (such as catalystic) requirements of the final product. DSM protein constructs with DSM that will not react with each others can be mixed together without them forming protein complexes. DSM protein constructs or other linker molecules can be added later to form gels or polymers.

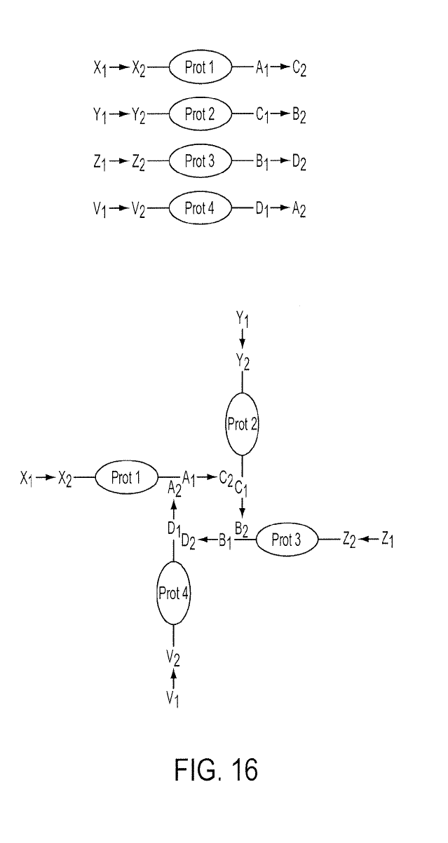

FIG. 16 shows four unique DSM protein constructs that can be used to build a four DSM protein construct junction, such as shown in FIG. 16, each with one unique, junction DSM. The approach is the same as above except with a fourth DSM protein construct with its own custom junction DSM. In fact, a junction of any number of DSM protein constructs can be created. If a junction of X DSM protein constructs is desired, one needs to design X DSM protein constructs, each with a unique junction DSM on one end. These junctions result in a roughly X-pointed star like structure with the junction DSMs joining in the center and the target proteins and other DSMs radiating. Randomly starting with one of the DSM protein constructs as the first, count clockwise (or counterclockwise) around the junction from protein construct #1 to the last protein construct, protein construct #X, which is adjacent to the first. For DSM protein construct W, one of the assembler protein parts of its junction DSM must be the complement of (domain swap with) one of the junction DSMs for the DSM protein construct that is adjacent to it in a counterclockwise direction (in position W-1), and the other of the assembler protein parts of the junction DSM of DSM protein construct W must be the complement of one of the junction DSMs for the DSM protein construct that is adjacent to it in a clockwise direction (in position W+1 for DSM protein construct W). Also, the second DSM, if there is one, for each of the X DSM protein constructs must not interact with any of the junction DSMs.

Whether these other DSMs are unique or can interact with one another is a design question. With the set of three protein constructs in FIG. 15, the DSM X₁-X₂ in construct #1 can domain swap with another protein construct #1 to connect two three protein structures from FIG. 15. The same is true of constructs #2 and #3, and with the four DSM protein complexes in FIG. 16, except that they will connect four-protein-construct structures such as that shown in FIG. 16.

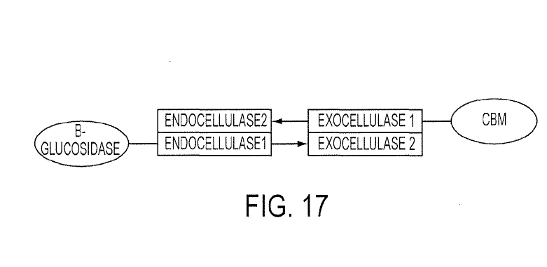

FIG. 17 shows domain swapping by two single-DSM protein constructs, where the target protein of one is Beta-glucosidase and the target protein of the other is a CBM. The DSM of each is composed of a part of endocellulase and a part of exocellulase with a lever protein between them, so that when the two DSMs domain swap, they form endocellulase and exocellulase. The protein complex formed is a polymer with four active proteins with linkers between: beta-glucosidase, endocellulase, exocellulase and CBM. Other proteins can be used instead of endocellulase and exocellulase as assembler proteins, provided they can split into two parts that will domain swap (it is unclear at this time whether endo- and exocellulase will be able to be used as assembler proteins). The target proteins can be switched with the assembler proteins in a structure such as this since the result of the domain swapping is a defacto target protein.

Although the present invention has been described in connection with a preferred embodiment, it should be understood that modifications, alterations, and additions can be made to the invention without departing from the scope of the invention as defined by the claims.

What is claimed is:

1. A first chimeric protein, wherein an amino acid sequence of a first protein is inserted into an amino acid sequence of a surface loop of a second protein, wherein said amino acid sequence of said first protein splits said second protein into an N-segment and a C-segment.

2. The chimeric protein of claim 1, wherein said N-segment and said C-segment are kept from assembling by said amino acid sequence of said first protein.

3. The chimeric protein of claim 1, further comprising:
a second chimeric protein comprising substantially the same sequence as the first chimeric protein, wherein the N-segment of said first chimeric protein assembles with the C-segment of said second chimeric protein.

4. The chimeric protein of claim 3, further wherein the C-segment of said first chimeric protein assembles with the N-segment of said second chimeric protein.

5. The chimeric protein of claim 1, wherein an N-to-C terminal length of said amino acid sequence of said first protein is at least twice as long as the distance between C-alpha atoms of the two amino acids that define the termini of the surface loop of said second protein.

6. The chimeric protein of claim 3, wherein said assembly occurs in response to a trigger.

7. The chimeric protein of claim 6, wherein the amino acid sequence of the first protein comprises a thermodynamic stability equal to or greater than a thermodynamic stability of the amino acid sequence of the second protein.

8. The chimeric protein of claim 7, wherein said thermodynamic stability of the amino acid sequence of the first protein in the presence of said trigger is equal to or greater than the thermodynamic stability of the amino acid sequence of the second protein.

9. The chimeric protein of claim 8, wherein the amino acid sequence of said first protein is modified to adjust the thermodynamic stability of the amino acid sequence of the first protein, such that the thermodynamic stability of the amino acid sequence of the first protein in the presence of said trigger is equal to or greater than the thermodynamic stability of the amino acid sequence of the second protein.

10. The chimeric protein of claim 1, wherein said first protein is selected from the group consisting of: ubiquitin, a ubiquitin-like protein, ASPP2, an isoform of ASPP2, and GCN4.

11. The chimeric protein of claim 1, wherein said second protein is selected from the group consisting of: barnase, ubiquitin, and ribose binding protein.

12. The chimeric protein of claim 1, wherein the C-segment of said second protein is selected from the group consisting of: RBP 36-110, RBP 61-277, RBP 127-277, RBP 212-277, RBP 260-277, Ub 20-76, Ub 37-76, Ub 64-76, barnase 23-110, barnase 37-110, barnase 48-110, barnase 67-110, barnase 80-110, and barnase 104-110.

13. The chimeric protein of claim 1, wherein the N-segment of said second protein is selected from the group consisting of: RBP 1-35, RBP 1-60, RBP 1-126, RBP 1-211, RBP 1-259, Ub 1-19, Ub 1-36, Ub 1-63, barnase 1-22, barnase 1-36, barnase 1-47, barnase 1-66, barnase 1-79, and barnase 1-103.

14. The chimeric protein of claim 1, wherein the C-segment of said second protein is an amino acid sequence of a third protein.

15. An isolated nucleic acid that encodes a chimeric protein of claim 1.

16. A multi-protein complex comprising at least two of the chimeric protein of claim 1.

17. The multi-protein complex of claim 16, wherein the complex comprises at least one loop.

18. The multi-protein complex of claim 16, wherein one of said four chimeric proteins comprises an amino acid sequence from CBM, one of said chimeric proteins comprises an amino acid sequence from endocellulase, one of said chimeric proteins comprises an amino acid sequence from exocellulase, and one of said chimeric proteins comprises an amino acid sequence from β-glucosidase.

19. A method of creating the chimeric protein of claim 1, comprising the steps of:
culturing a plurality of host cells comprising at least one expression vector encoding for the first chimeric protein, under conditions sufficient for expression of said first chimeric protein; and
isolating said first chimeric protein produced by said host cells.

20. A polymer comprising a first chimeric protein of claim 1 bound at its C-terminal end to the N-terminal end of a fourth protein, or bound at its N-terminal end to the C-terminal end of the fourth protein.

21. The polymer of claim 20, wherein the free terminal end of the fourth protein, C- or N-, is bound to the appropriate terminal end, N- or C-, of a second chimeric protein of claim 1.

22. A protein complex comprising a branched hydrogel of the chimeric protein of claim 1.

23. A method of creating the polymer of claim 20, comprising the steps of:
culturing a plurality of host cells comprising at least one expression vector encoding for the first chimeric protein, under conditions sufficient for expression of said first chimeric protein; and
isolating said first chimeric protein produced by said host cells.

24. A method for creating a protein complex, comprising the step of combining a first polymer according to claim 20 with a second polymer according to claim 20, wherein either the C- or N-segment of a chimeric protein of said first polymer will assemble with either the C- or N-segment of a chimeric protein of said second polymer.
